(19) United States
(12) Patent Application Publication
(10) Pub. No.: US 2010/0228269 A1
(43) Pub. Date: Sep. 9, 2010

(54) VESSEL CLOSURE CLIP DEVICE

(76) Inventors: Michi E. Garrison, Half Moon Bay, CA (US); Richard J. Renati, Los Gatos, CA (US); Alan K. Schaer, San Jose, CA (US); Gregory M. Hyde, Menlo Park, CA (US)

Correspondence Address: MINTZ, LEVIN, COHN, FERRIS, GLOVSKY AND POPEO, P.C, ONE FINANCIAL CENTER, BOSTON, MA, 02111, US

(21) Appl. No.: 12/713,630

(22) Filed: Feb. 26, 2010

Related U.S. Application Data
(60) Provisional application No. 61/156,367, filed on Feb. 27, 2009.
(60) Provisional application No. 61/181,588, filed on May 27, 2009.

(51) Int. Cl. A61B 17/128; A61B 17/122

(52) U.S. Cl. 606/139; 606/158

(57) ABSTRACT

A clip-based vascular closure devices is configured to be pre-applied to a blood vessel prior to insertion of a vascular access device (such as a procedural sheath) through an incision, puncture, penetration or other passage through the blood vessel. In an embodiment, the disclosed closure device is applied in a carotid artery via a transcervical access such as by forming an incision in the patient's neck to in order to access the blood vessel or other body lumen.

### CROSS-REFERENCES TO PRIORITY DOCUMENTS

This application claims priority of co pending U.S. Provisional Patent Application Ser. No. 61/156,367 filed on Feb. 27, 2009 and U.S. Provisional Patent Application Ser. No. 61/181,588 filed on May 27, 2009. Priority of the aforementioned filing dates is hereby claimed and the disclosures of the provisional patent applications are hereby incorporated by reference in their entirety.

### BACKGROUND

The present disclosure relates generally to medical methods and devices. More particularly, the present disclosure relates to methods and devices for closure of puncture wounds into vessels wherein the closure devices are sometimes applied before the vessel is accessed with a sheath or cannula.

Medical procedures for gaining intravascular arterial access are well-established, and fall into two broad categories: surgical cut-down and percutaneous access. In a surgical cut-down, a skin incision is made and tissue is dissected away to the level of the target artery. Depending on the size of the artery and of the access device, an incision is made into the vessel with a blade, or the vessel is punctured directly by the access device. In some instances, a micro-puncture technique is used whereby the vessel is initially accessed by a small gauge needle, and successively dilated up to the size of the access device.

For percutaneous access, a puncture is made from the skin, through the subcutaneous tissue layers to the vessel, and into the vessel itself. Again, depending on the size of the artery and of the access device, the procedure will vary, for example a Seldinger technique, modified Seldinger technique, or micro-puncture technique is used.

Because arteries are high-pressure vessels, additional maneuvers may be required to achieve hemostasis after removal of the access device from the vessel. In the case of surgical cut-down, a suture may be used to close the arteriotomy. For percutaneous procedures, either manual compression or a closure device may be used. While manual compression remains the gold standard with high reliability and low cost, closure devices require less physician time and lower patient recovery time. In addition, closure devices are often required for procedures with larger access devices and/or for patients with anti-coagulation and anti-platelet therapy. Examples of closure devices include suture-based closure devices such as the Abbott Vascular Perclose or ProStar family of devices or the Sutura Stitch device; clip closure devices such as the Abbott Vascular StarClose device, or “plug” closure devices such as the Kensey Nash/JNJ AngioSeal device.

In certain types of procedures, it is advantageous to “pre-close” the arteriotomy, for example if the arteriotomy is significant in size, if the arteriotomy site is difficult to access, or if there is a heightened risk of inadvertent sheath removal. In the latter case, the ability to gain rapid hemostatic control of the access site can be critical. In an open surgical procedure, a suture is sometimes placed into the vessel wall in a U-stitch, Z-stitch, or purse-string pattern prior to vessel access. The arteriotomy is made through the center of this stitch pattern. The suture may be tensioned around the sheath during the case, or be left loose. Generally, the two ends of the suture exit the incision and are anchored during the procedure, for example with hemostatic forceps. If the sheath is inadvertently removed, rapid hemostasis may be achieved by applying tension to the ends of the suture. After device removal, the suture ends are then tied off to achieve permanent hemostasis.

In procedures with limited access to the arteriotomy, for example if the approach was percutaneous, or if the incision was small and/or if the patient was obese, it may be difficult to insert a closing suture in this manner. Furthermore, in instances where it is only possible to insert a short length of the access device, for example where the access site is very close to the target treatment site, there is a heightened risk of inadvertent device removal. A pre-applied device which can immediately or quickly achieve hemostasis when the device is removed offers some benefit. In addition, if the pre-applied device offered some resistance to removal force, the chance of inadvertent removal would be reduced.

The suture-based percutaneous closure devices noted above have been used to “pre-close.” These devices require entering the vessel with the deployment device to place the stitches. In the case of the Abbott ProStar, the vessel entry device requires about 15 cm length into the vessel. In instances where vascular space is limited, these types of devices are not feasible. Although the clip devices such as the StarClose device has been used for “re-access”, it has not been designed for this purpose. Elements on this type of device which are designed to seal the puncture may easily interfere with sheath insertion and/or removal, and may cause vessel trauma.

In certain clinical procedures, for example procedures requiring access to the carotid arteries, the consequences of failure of the vascular closure devices to achieve complete hemostasis are greater. In this instance, if the vessel closure device did not achieve full hemostasis, the resultant hematoma may lead to loss of airway passage and/or critical loss of blood to the brain, both of which lead to severe patient compromise and possibly death. If the vascular closure device contained intravascular elements which embolized, the embolic substance could enter the cerebral circulation and cause major brain injury.

### SUMMARY

In view of the foregoing, there are herein disclosed clip-based vascular closure devices that are configured to be pre-applied to a blood vessel prior to insertion of a vascular access device (such as a procedural sheath) through an incision, puncture, penetration or other passage through the blood vessel. The clip-based vascular closure devices can also be applied to the blood vessel after insertion of the vascular access device but before removal of the vascular access device, or after removal of the vascular access device. The closure devices can achieve rapid hemostasis upon either deliberate or inadvertent sheath removal. The disclosed devices require minimal entry into the vessel to be deployed. Furthermore, the devices leave minimal material or no material inside the vessel and have an extremely reliable means of achieving hemostasis, making the chance of a hematoma remote. In an embodiment, the disclosed closure device is applied in a carotid artery via a transcervical access such as by forming an incision in the patient's neck to in order to access the blood vessel or other body lumen.

In one aspect, there is disclosed a vessel closure device, comprising: an annular body defining a plane and disposed about a central axis at the center of an opening of the body, the body being movable from a generally planar configuration lying generally in the plane towards a generally cylindrical configuration extending out of the plane, the body comprising a plurality of looped elements comprising alternating first and second curved regions, the first curved regions defining an inner periphery of the body and the second curved regions defining an outer periphery of the body in the planar configuration; and a plurality of tissue attachment features extending from the first curved regions, the attachment features being oriented generally into the opening of the body in the planar configuration in a manner that does not interfere with insertion and removal of a procedural sheath through the opening, and generally parallel to the central axis in the transverse configuration.

In another aspect, there is disclosed a vessel closure device, comprising: an annular body defining a plane and disposed about a central axis at the center of an opening of the body, the body being movable from a generally expanded configuration towards a generally compressed configuration, wherein the body is spring biased toward the compressed configuration and wherein the body applies a generally linear force to tissue as the body moves toward the compressed configuration; and a plurality of tissue attachment features extending from the body for attaching to tissue.

In another aspect, there is disclosed a vessel closure device, comprising: an annular body with a central opening; a plurality of attachment features being oriented in a manner that does not interfere with insertion and removal of a procedural sheath through the opening; and a self-sealing member attached to the body for sealing an opening in the vessel.

In another aspect, there is disclosed a vessel closure device, comprising: an annular body defining a plane and disposed about a central axis at the center of an opening of the body, the body being movable from a generally planar configuration lying generally in the plane towards a generally cylindrical configuration extending out of the plane, the body comprising a plurality of looped elements comprising alternating first and second curved regions, the first curved regions defining an inner periphery of the body and the second curved regions defining an outer periphery of the body in the planar configuration; a plurality of posts extend from the annular body in a cork-screw configuration; a seal member on the posts for sealing an opening in the vessel; and a plurality of attachment features extending from the first curved regions, wherein the posts fold as the annular body transitions from the cylindrical configuration to the planar configuration in a manner that causes the seal member to collapse in a contractile circular manner over the opening in the tissue.

In another aspect, there is disclosed a vessel closure device, comprising: at least one clip with at least one attachment feature that attaches to tissue; and at least one closing suture pre-attached to the clip, wherein the closing suture can be tightened to cause the clip to collapse and thereby close the an opening in the tissue to which the clip is attached

In another aspect, there is disclosed a vessel closure device, comprising: an annular body with a central opening; a plurality of attachment features being oriented in a manner that does not interfere with insertion and removal of a procedural sheath through the opening; and a seal member attached to the body for sealing an opening in the tissue, the seal member being movable from a first position that does not interfere with the opening in the annular body and a second position that extends over the opening and seals an opening in the vessel.

In another aspect, there is disclosed a vessel closure device, comprising: an annular body with at least one attachment feature that attaches to tissue; a seal member fastened to the annular body for sealing an opening in the tissue; and a fastener element integral to the annular body that fastens the seal to the tissue.

In another aspect, there is disclosed a vessel closure device delivery system, comprising: a delivery device that couples to a vessel closure clip for delivering the clip onto a blood vessel; and a suction element coupled to the delivery device, the suction element adapted to apply suction to a wall of the blood vessel when the delivery device is delivering the clip; and a vessel closure clip.

In another aspect, there is disclosed a vessel closure device delivery system, comprising: a delivery device that couples to a vessel closure clip for delivering the clip onto a blood vessel; a retractable vessel locator removeably attached to the delivery device, the distal end of the vessel locator adapted to transition from a collapsed state suitable for insertion into a vessel and an expanded state that lodges against a wall of the vessel from inside the vessel; and a vessel closure clip

In another aspect, there is disclosed a vessel closure device delivery system, comprising: a delivery device that couples to a vessel closure clip for delivering the clip onto a blood vessel; a procedural sheath that couples onto the delivery device such that the procedural sheath can be advanced over or through the delivery device; and a vessel closure clip.

In another aspect, there is disclosed a vessel closure device delivery system, comprising: a delivery device that couples to a vessel closure clip for delivering the clip onto a blood vessel; a counter traction element that prevents the clip from being detached from the blood vessel during removal of the delivery device; and a vessel closure clip.

In another aspect, there is disclosed a system of devices for treating carotid or cerebral artery disease or the brain, comprising: a vessel closure clip; a delivery device that couples to the vessel closure clip for delivering the clip onto a blood vessel; and an arterial access sheath adapted to be introduced into a common carotid, internal carotid, or vertebral artery through a penetration in the artery and receive blood from the artery, wherein the arterial access sheath couples onto the delivery device such that the arterial access sheath can be advanced over or through the delivery device.

In another aspect, there is disclosed a method for closing an opening in a wall of a body lumen, comprising: placing a clip on the wall of the body lumen; advancing a procedural sheath through the clip into the body lumen; and inserting a procedural device through the procedural sheath into the body lumen.

In another aspect, there is disclosed a method for closing an opening in a wall of a body lumen, comprising: providing a procedural sheath having a vessel closure clip pre-mounted on the procedural sheath; placing the procedural sheath through the wall of the body lumen; inserting a procedural device through the sheath into the body lumen; performing a procedure using the procedural device; advancing the vessel closure clip; and removing the procedural sheath from the clip and the body lumen.

In another aspect, there is disclosed a method for closing an opening in a wall of a body lumen, comprising: providing a vessel closure clip delivery device with a pre-mounted procedural sheath; placing a clip on the wall of the body lumen; advancing the procedural sheath through the clip and through the wall of the body lumen; inserting a procedural device through the sheath into the body lumen; performing a procedure using the procedural device; removing the procedural sheath from the clip and the body lumen.

In another aspect, there is disclosed a method for performing a procedure on a carotid or cerebral artery, comprising: inserting a procedural sheath through the wall of the common carotid artery; occluding the common carotid artery; inserting a procedural device through the procedural sheath into the common carotid artery and performing a procedure on the carotid or cerebral artery; removing the procedural sheath; and placing a vessel closure clip on the wall of the artery to close the access site of the common carotid artery.

In another aspect, there is disclosed a method for closing an opening in a wall of a body lumen, comprising: placing a clip on a penetration that extends through the wall of the body lumen; advancing a procedural sheath through the penetration into the body lumen; and inserting a procedural device through the procedural sheath into the body lumen.

Other features and advantages should be apparent from the following description of various embodiments, which illustrate, by way of example, the principles of the invention.

### BRIEF DESCRIPTION OF THE DRAWINGS

FIG. 1 shows an example of a closure device.

FIG. 2A shows another embodiment of a closure device.

FIG. 2B shows a perspective view of the closure device of FIG. 2A during deployment.

FIG. 2C shows the closure device of FIG. 2A mounted on a delivery system.

FIGS. 3, 4, 5A, and 5B show alternate embodiments of closure devices.

FIGS. 6A and 6B show a schematic representation of an arteriotomy comprising an incision.

FIGS. 7A-7B show a first embodiment of a closure device that applies linear closing forces.

FIGS. 8A-8B show another embodiment of a closure device that applies linear closing forces.

FIGS. 9A and 9B show an embodiment of a sealing closure device.

FIG. 10 shows another embodiment of a sealing closure device.

FIGS. 11A-11C show another embodiment of a sealing closure device.

FIGS. 12A, 12B, 13, and 14 show embodiments of a pre-tied closure device.

FIGS. 15A-15C show an embodiment of a combination closure device that combines a closure clip with a spring-loaded sealing element.

FIGS. 16A-16D show another embodiment of a closure device that includes an upper clip member positioned over a lower clip member and trapping a sealing member.

FIGS. 17A-17D and 18 show additional embodiments of combination closure devices.

FIGS. 19A-19C show a closure device with an exemplary delivery device.

FIGS. 20A-20C show another embodiment of a closure device.

FIGS. 21A-21B show a suction delivery system that is used to deliver a closure device.

FIGS. 22A-22B show a locating device that can be used in conjunction with delivery of a closure device.

FIGS. 23A-23C show an example of a closure device pre-mounted on a procedural sheath such that the procedural sheath serves as a central delivery shaft of the delivery system.

FIGS. 24A-24C show an example of the procedural sheath mounted on the central delivery shaft of the delivery system.

FIG. 25 shows a tube located on the outside of a delivery sheath.

FIG. 26 shows an exemplary embodiment of a retrograde flow system 100 that is adapted to establish and facilitate retrograde or reverse flow blood circulation.

FIG. 27 shows an interventional device being introduced into the carotid artery via an arterial access device.

FIG. 28A illustrates an arterial access device useful in the methods and systems of the present disclosure.

FIG. 28B illustrates an additional arterial access device construction with a reduced diameter distal end.

FIGS. 29A and 29B illustrate a tube useful with the sheath of FIG. 10A.

FIG. 30A illustrates an additional arterial access device construction with an expandable occlusion element.

FIG. 30B illustrates an additional arterial access device construction with an expandable occlusion element and a reduced diameter distal end.

FIG. 31 illustrates a first embodiment of a venous return device useful in the methods and systems of the present disclosure.

FIG. 32 illustrates an alternative venous return device useful in the methods and systems of the present disclosure.

FIG. 33 shows an example of the reverse flow system with a schematic representation of the flow control assembly.

FIG. 34A-34D, FIGS. 35A-35D, FIGS. 36A and 36B, FIGS. 37A-37D, and FIGS. 38A and 38B, illustrate different embodiments of a variable flow resistance component useful in the methods and systems of the present disclosure.

FIGS. 39A-39B, FIGS. 40A-40B, FIGS. 41A-41D, and FIGS. 42A-42B illustrate further embodiments of a variable flow resistance system useful in the methods and systems of the present disclosure.

FIGS. 43A-43E illustrate exemplary blood flow paths during an exemplary procedure for implanting a stent at the carotid bifurcation.

FIG. 44A shows an arterial access sheath with a proximal extension

FIG. 44B shows the proximal extension removed from the arterial access sheath of FIG. 44A.

### DETAILED DESCRIPTION

Disclosed herein are clip-based vascular closure devices that are configured to be pre-applied to a blood vessel prior to insertion of a vascular access device (such as a procedural sheath) through an incision, puncture, penetration or other passage through the blood vessel. The clip-based vascular closure devices can also be applied to the blood vessel after insertion of the vascular access device but before removal of the vascular access device, or after removal of the vascular access device. The closure devices can achieve rapid hemostasis upon either deliberate or inadvertent sheath removal. The disclosed devices require minimal entry into the vessel to be deployed. Furthermore, the devices leave minimal material or no material inside the vessel and have an extremely reliable means of achieving hemostasis, making the chance of a hematoma remote. In an embodiment, the disclosed closure device is applied in a carotid artery via a transcervical access such as by forming an incision in the patient's neck to in order to access the blood vessel or other body lumen.

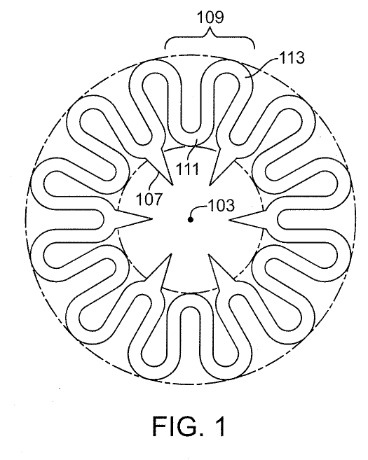

An existing closure device is described in U.S. Pat. No. 6,623,510 and an embodiment is shown in FIG. 1. U.S. Pat. No. 6,623,510 is incorporated herein by reference in its entirety. The existing closure device is comprised of a clip for closing an incision, puncture, penetration, or other passage through a blood vessel or other body lumen. The clip is adapted to transition between a cylindrical configuration and a flat or planar configuration, as described more fully below. FIG. 1 shows the clip in the planar configuration The clip includes a body, which may be generally annular in shape and which surrounds a central axis 103. The central axis 103 extends outward normal to the plane of FIG. 1 and may be at the center of a central opening of the body. The clip further includes a plurality of attachment features such as tines 107 extending from the body. The tines 107 extend along an axis that intersects or abuts the central axis 103. U.S. patent application Ser. No. 11/356,214, U.S. patent application Ser. No. 10/638,115, U.S. patent application Ser. No. 11/048,503, U.S. patent application Ser. No. 11/427,297, U.S. patent application Ser. No. 12/143,020, U.S. patent application Ser. No. 10/356,214, U.S. patent application Ser. No. 10/638,115, U.S. patent application Ser. No. 11/048,503, U.S. patent application Ser. No. 11/427,297, and U.S. patent application Ser. No. 12/143,020 describe exemplary closure devices and delivery systems. These applications are incorporated by reference in their entirety.

The annular body may include a plurality of looped or curved elements 109 that are connected to one another to form the body. Each looped element 109 may include an inner or first curved region 111 and an outer or second curved region 113. In an embodiment, the first and second curved regions 111, 113 are out of phase with one another and are connected alternately to one another, thereby defining an endless sinusoidal pattern. When the clip is in the substantially flat or planar configuration, as shown in FIG. 1, the first curved regions 111 may define an inner periphery of the body and the clip, and the second curved regions 113 may define an outer periphery of the body. A disadvantage of the clip shown in FIG. 1 and the clips described in U.S. Pat. No. 6,623,510 is that the tines 107 of the clip are arranged in a manner that tends to interfere with passage of a vascular access device such as a procedural sheath through the center of the clip.

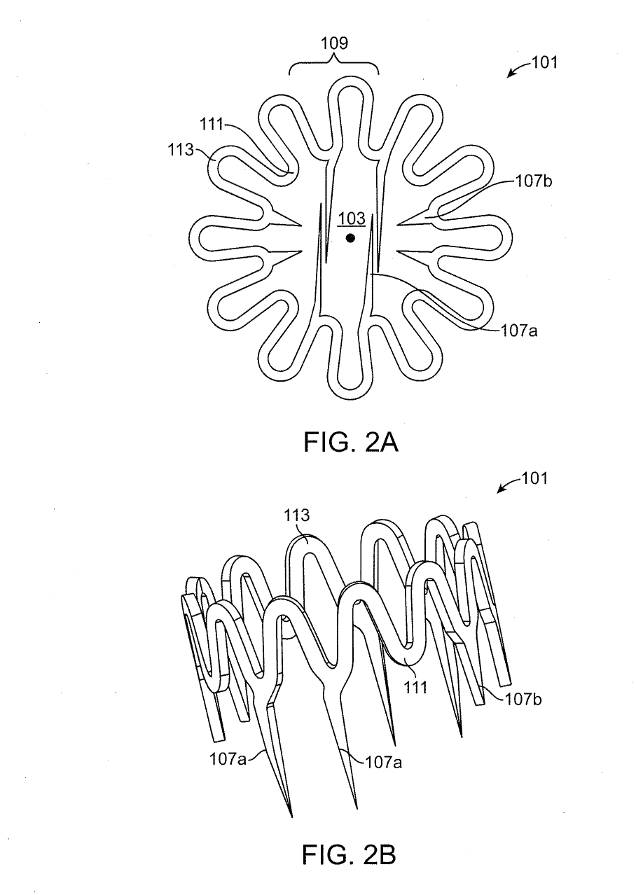

FIG. 2A shows an improved embodiment of a closure device comprised of a clip 101. The annular body of the clip 101 has a central opening that is configured to receive a procedural sheath that can be inserted into a blood vessel, as described more fully below. The tines 107 are arranged in a manner such that they do not interfere with, impede or interrupt insertion and/or removal of the procedural sheath through the body. The body can include any hollow body, for example, including one or more structures surrounding an opening, whether the body is substantially flat or has a significant thickness or depth. Thus, although an annular-shaped body may be circular, it may include other noncircular shapes as well, such as elliptical or other shapes that are asymmetrical about a central axis.

The plurality of tines 107 are biased to extend generally inwardly towards one another and such that the tines do not intersect the central axis 103. Thus, the tines 107 extend along an axis that is offset or angled away from the central axis 103. The tines 107 may be disposed on the first curved regions 111 generally toward the body's central region but not intersecting the central axis 103 when the clip 101 is in the planar configuration. In an embodiment, the tines 107 may be provided in pairs opposite from one another or provided otherwise symmetrically with respect to the central axis 103.

In the embodiment of FIG. 2A, the tines 107 include one or more major tines 107a that are of a longer length as well as one or more minor tines 107b that are shorter in length than the major tines 107a. The major tines 107a extend along an axis that is offset a distance from intersection with the central axis 103. For example, the major tines 107a may be offset a distance of 0.010″ to 0.030″ from the central axis 103. Such a configuration minimizes or eliminates interference with the sheath that is inserted through the center of the body. For example, two pairs of major tines 107a extend inwardly toward the center of the body but offset from the central axis 103. The longer length of the major tines 107a make them more likely to interfere with passage of the procedural sheath through the body so it is desirable that the major tines 107a have a maximum amount of offset from the central axis 103 while still preserving the function of compressing the vessel wall around the area of an arteriotomy to provide hemostasis after removal of the access device.

As shown in FIG. 2B, the annular body and/or the tines 107 may be deflected into a cylindrical configuration such that the tines 107 are oriented parallel to the central axis 103 and the body may have a generally annular shape having a length that extends generally parallel to the central axis 103, and corresponds generally to an amplitude of the zigzag pattern. The body may be sufficiently flexible so that the clip 101 may assume a generally circular or elliptical shape, such that it can be placed around the exterior surface of a central shaft 606 of a delivery system, as shown in FIG. 2C. As discussed below, the central shaft of the delivery system can be a procedural sheath or other vascular access device.

In an embodiment, the tines 107 and/or body are biased to move from the cylindrical configuration (shown in FIG. 2B) towards the planar configuration (shown in FIG. 2A). Thus, with the tines 107 in the cylindrical configuration, the tines 107 may penetrate and/or be engaged with tissue at a puncture site. When the clip 101 is released, the tines 107 may attempt to return towards one another as the clip 101 moves towards the planar configuration, thereby drawing the engaged tissue together and substantially closing and/or sealing the puncture site, as explained further below.

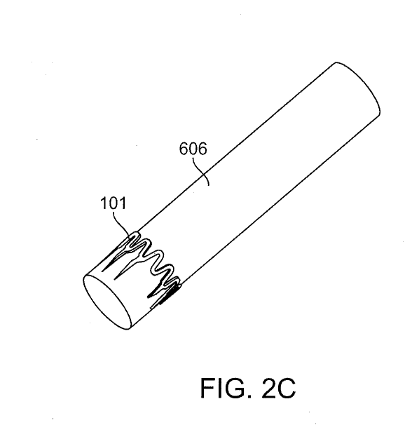

In another embodiment shown in FIG. 3, the loops 109a around two opposing sections of the clip 101 are thinner than the remainder of the loops 109b. Thus, the loops 109a deform more easily than the loops 109b. In this embodiment, the clip acts as a spring with a closing force is not radially uniform but rather directed linearly towards the arteriotomy.

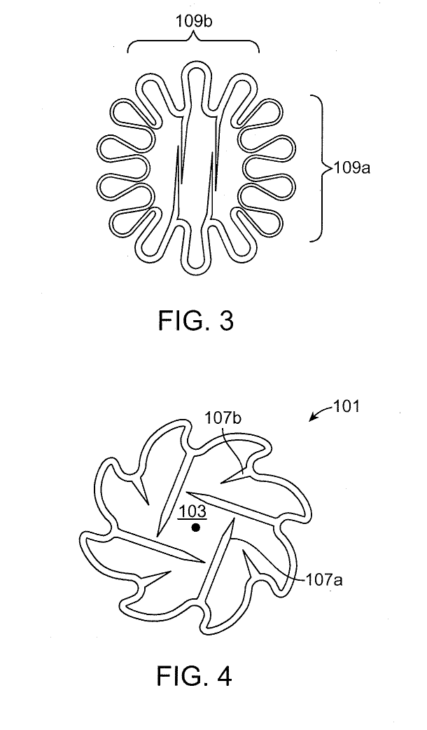

FIG. 4 shows another embodiment wherein all of the tines 107 (including the major tines 107a and minor tines 107b) extend along respective axes that do not intersect the central axis 103. In the planar configuration, at least one of the tines extends along an axis that intersects an axis of another tine. None of the axes of the attachment features intersect the central axis. The tines 107 point off-center from the central point 103 of the opening when the device is in the planar configuration. In this manner, the tines 107 are arranged in an iris-like configuration around the central axis 103. This configuration reduces the likelihood that the tines 107 will interfere with the sheath during insertion and removal through the central axis 103.

The tines 107 may include a variety of pointed tips, such as a bayonet tip, and/or may include barbs for penetrating or otherwise engaging tissue. For example, to increase the penetration ability of the clip 101 and/or to lower the insertion force required to penetrate tissue, each tine 107 may include a tapered edge extending towards the tip along one side of the tine 107. Alternatively, each tine 107 may be provided with a tapered edge on each side of the tine 107 extending towards the tip.

Additionally, the tines 107 may be disposed on alternating first curved regions 111. Thus, at least one period of a zigzag pattern may be disposed between adjacent tines 107, which may enhance flexibility of the clip 101.

The looped elements 109 may distribute stresses in the clip 101 as it is deformed between the cylindrical and the planar configurations, thereby minimizing localized stresses that may otherwise plastically deform, break, or otherwise damage the clip 101 during delivery. To manufacture the clip 101 (or, similarly, any of the other clips described herein), the body and the tines 107 may be integrally formed from a single sheet of material, e.g., a superelastic alloy, such as a nickel-titanium alloy (“Nitinol”). Portions of the sheet may be removed using conventional methods, such as laser cutting, chemical etching, photo chemical etching, stamping, using an electrical discharge machine (EDM), and the like, to form the clip. The tines 107 may be sharpened to a point, i.e., tips may be formed on the tines 107 using conventional methods, such as machining, mechanical grinding, and the like.

The clip 101 may be polished to a desired finish using conventional methods, such as electro-polishing, chemical etching, tumbling, sandblasting, sanding, and the like. Polishing may perform various functions depending on the method used to form the clip 101. For a clip formed by laser cutting or using an EDM, polishing may remove heat affected zones (HAZ) and/or burrs from the clip. For a clip formed by photo chemical etching, polishing may create a smoother surface finish. For a clip formed by stamping, polishing may remove or reduce burrs from the bottom side of the clip, and/or may smooth the “roll” that may result on the topside of the clip from the stamping process.

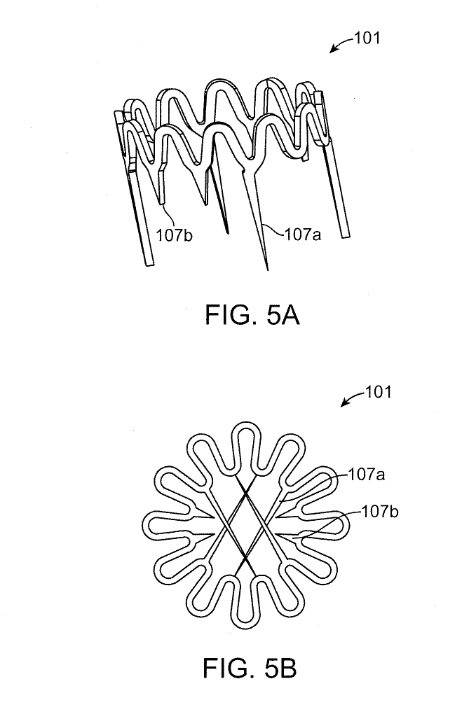

FIGS. 5A and 5B show another embodiment of the clip 101 in the cylindrical and planar states, respectively. In this embodiment, the major tines 107a have an increased length with respect to the previous embodiments. The increased length of the major tines 107a reduces the likelihood that the major tines 107a will interfere with a sheath as the tines would tend to deflect to one or the other side of the sheath as the sheath is inserted through the central axis 103. Because the tines overlap in the planar configuration in this embodiment (as shown in FIG. 5B), the clip must be manufactured from a tube rather than a flat sheet, as shown in FIG. 5A. After the cutting and polishing process is complete, the clip is flattened and heat set to the planar state of FIG. 5B, such as by using Nitinol processing methods well known in the art. The annular body may also include one or more upwardly extending bars (not shown) that can be used to assist in flattening the clip to the planar state during the flattening and heat-set process. The bars may be removed from the clip after the heat set process is complete.

### Linear Compressive Spring Embodiment

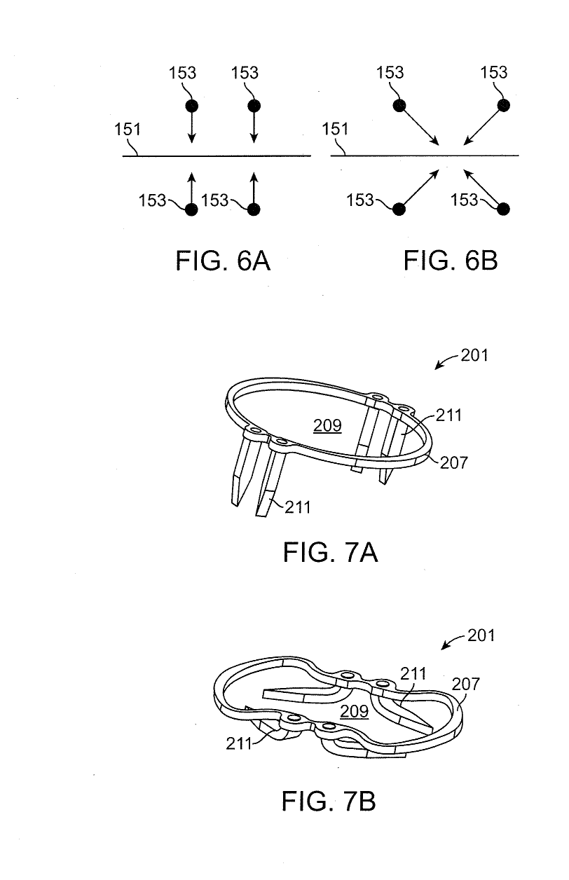

Additional clip embodiments are now described wherein the clip provides closure force(s) that are linear across the pathway of the arteriotomy in the same or similar manner that a suture would apply closing forces. FIGS. 6A and 6B show a schematic representation of an arteriotomy comprising an incision 151. The arrows show the direction of force caused by conventional suture closure. FIG. 6A shows the forces created by two interrupted sutures, and FIG. 6B shows the force of a suture placed in a Z-configuration. The following clip embodiments recreate these forces on the arteriotomy. In a first embodiment, the clip applies closure forces that are directed linearly across the incision 151, as in FIG. 6A. In another embodiment, the clip applies closure forces along vectors that intersect one another as in FIG. 6B. The attachment locations 153 of the clip to the blood vessel tissue are positioned out of the entry pathway of the procedural sheath as the sheath enters the blood vessel. This minimizes interference of the clip with the sheath.

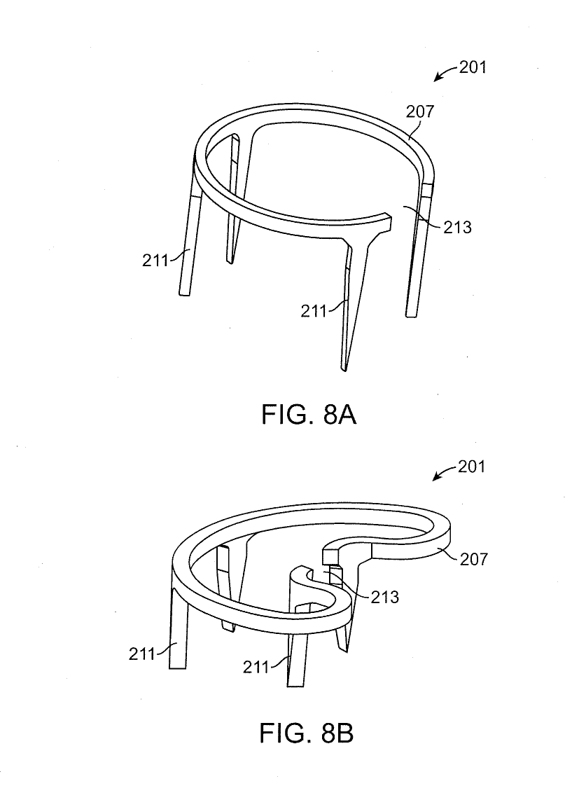

FIGS. 7A-7B show a first embodiment of the clip 201 that applies linear closing forces as described above. That is, the clip has a spring force that closes the annular body of the clip from the cylindrical configuration to the planar configuration pursuant to a generally linear rather than radial bias. The clip 201 includes an annular body comprised of a ring member 207, and a set of attachment tines 211 that are configured to be positioned on either side of the arteriotomy such as in the arrangement of the attachment locations 153 shown in FIGS. 6A and 6B. The ring member 207 is of an annular or partially annular configuration in that it surrounds a central opening 209 for receipt of the procedural sheath. The ring member 207 is biased from an expanded state toward a radially inward state or compressed state relating to a decreased size of the opening 209 to provide a closing force to the arteriotomy when the procedural sheath or delivery shaft is removed. As shown in FIG. 7A, during delivery the clip 201 is fully expanded such that the ring member 207 is round or substantially round, and the attachment tines 211 are constrained to be pointing downwards. The clip is biased inward. When the delivery shaft is removed from the center of the clip, the clip collapses inward and the attachment tines 211 deflect to their biased state parallel to the vessel wall to anchor the clip, as shown in FIG. 7B. As the clip 201 collapses, the clip 201 provides a linear closing force to the arteriotomy. That is, the tines move toward one another in pairs along a linear vector, such as in the manner shown in FIG. 6A or 6B. The tines thereby draw the arteriotomy closed. A procedural sheath is then inserted through the clip such that the clip re-expands to accept the sheath. When the procedural sheath is removed, the clip reverts to its biased, radially inward state to provide a closing force on the arteriotomy.

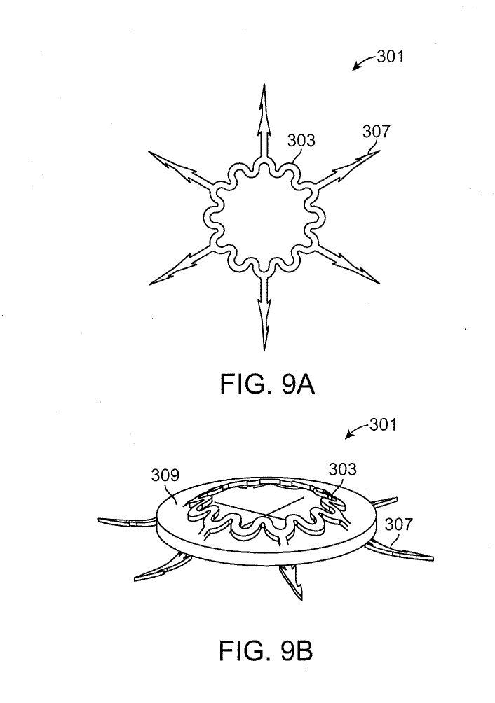

FIGS. 8A and 8B show another embodiment wherein the linear closure clip 201 is of a partially annular configuration. FIG. 8A shows the clip 201 with the ring member 207 in an expanded or non-collapsed state. In this embodiment, the ring 207 is not fully enclosed but rather has an opening 213 that permits the ring 207 to collapse, as shown in FIG. 8B. It should be appreciated that variations on the configuration of the ring 207 are possible.

### Seal Attachment Embodiments

In another embodiment of the closure device, a seal member is pre-attached to a clip. The clip attaches to the blood vessel via tines and provides a closure force to the arteriotomy. In conjunction with the closure force provided by the clip, the seal member acts as a compressive seal to the arteriotomy. The seal may be pre-cut and/or a self-sealing material.

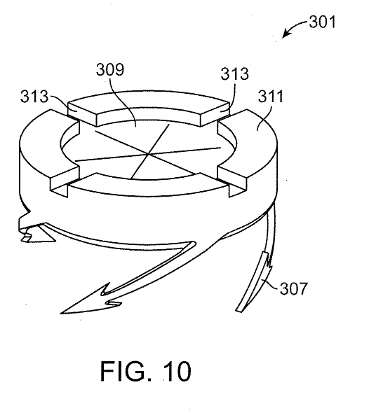

FIG. 9A shows a first embodiment of a sealing clip 301 comprised of an annular body formed of a central ring 303, and a plurality of tines 307. The central ring 303 has an opening through which the procedural sheath can be inserted. As shown in FIG. 9B, a seal member 309 is coupled to the clip 301 such that the ring 303 inserts through the seal member 309 via the tines 307. The seal member 309 can have a pre-cut opening that permits the procedural sheath to be inserted through the seal member 309. In use, the sealing clip 301 flattens when deployed onto the blood vessel wall and splays outward into the vessel wall, as shown in FIG. 9B. In this manner, the central ring 303 and tines 307 provide a closing force to the arteriotomy while anchoring the seal member to the vessel wall, while the seal member 309 provides additional sealing force to the arteriotomy.

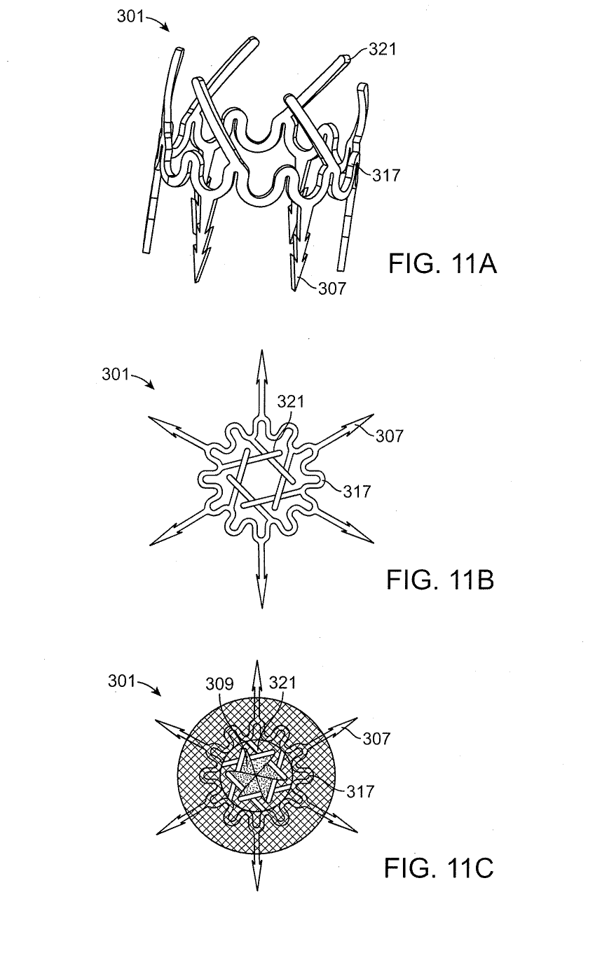

FIG. 10 shows another embodiment of a sealing clip 301. In this embodiment, the sealing clip 301 includes an annular body 311 having one or more tines 307 extending therefrom. The tines 307 may be arranged in a manner that permits them to be screwed into the tissue of the vessel. For example, the tines 307 can be arranged in a spiral or “cork-screw” configuration. The annular body 307 can include one or more engagement features 313, such as one or more slots or other engagement features, that can be coupled to a torquing tool. The tool can then be used to apply a rotational force to the annular body 311 for screwing the clip into the vessel wall.

A seal member 309 is coupled to the annular body 311. The seal member 309 can have a pre-cut opening that permits the procedural sheath to be inserted through the seal member 309 and through the center of the annular body 311. The seal member material and design in relation to the annular body are configured such that the seal is “self-sealing”. In other words when the delivery device or procedural sheath is removed from the central opening, the seal member provides a hemostatic seal over the arteriotomy. For example, the seal member material may be a soft elastomer such as silicone rubber or polyurethane and the seal member may be in a slight compressed state when assembled in the annular body. As in the previous embodiment, the annular body 311 and tines 307 attach the seal member to the vessel wall, while the seal member 309 seals the arteriotomy.

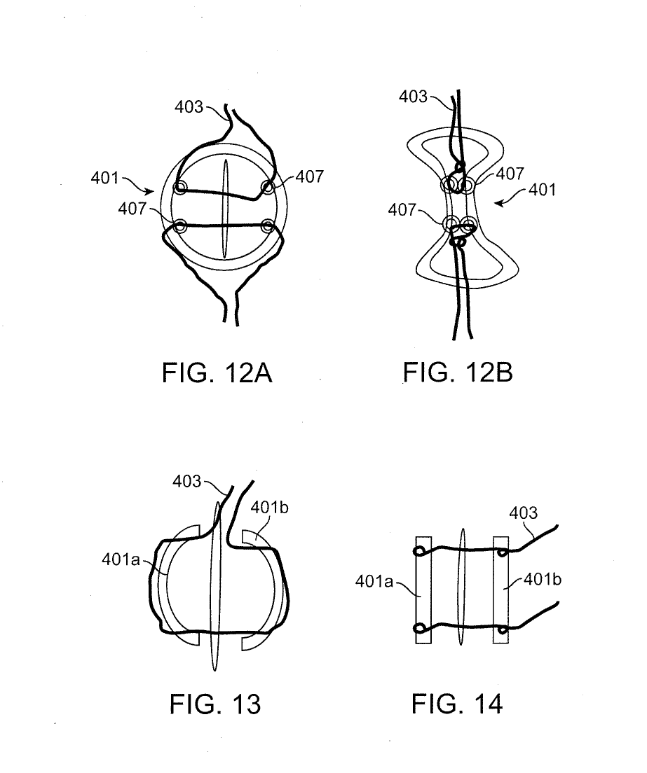

FIG. 11A shows another embodiment of a sealing clip 301. In this embodiment, the sealing clip 301 includes an annular body 317 having one or more tines 307 extending therefrom. The annular body 317 has a similar configuration to the undulating loop annular body described above with reference to FIG. 2 although it should be appreciated that the configuration of the annular body can vary. A plurality of upwardly-extending posts 321 extend from the annular body and are arranged in a spiral or cork-screw configuration. A seal member can be positioned on the posts for sealing the arteriotomy. As described below, the sealing clip 301 collapses when deployed such that the posts 321 collapse and fold in an iris pattern over the arteriotomy. That is, the posts 321 cause the seal to close in a circular, contractile manner.

The clip 301 of FIG. 11A can be manufactured by cutting it out of a tube such that it has the configuration shown in FIG. 11A. The clip 301 can then be flattened to the achieve the configuration shown in FIG. 11B such that the tines 307 splay outward into the vessel wall. When flattened, the spiral arrangement of the posts 321 causes them to fold over one another in an iris fashion such that they fold over the arteriotomy.

FIG. 11C shows the clip 301 in the planar state with the seal member 309 mounted on the clip. The seal member 309 is mounted over the clip 301 such that a region of the seal member 309 is coupled to the posts 321. As the posts 321 fold downward, they pull the seal member 309 over the arteriotomy. The seal member 309 folds over itself to create a compressive iris-style seal. The iris-style seal may be stretched open during insertion of a procedural sheath through the central opening, and then re-seal over the arteriotomy once the sheath is removed.

### Pre-Tied Closure Clips

In another embodiment, a clip has a pre-attached suture. The clip attaches to the vessel wall in a pattern around the arteriotomy location, for example with deflectable attachment tines as shown in FIGS. 7A and 7B. The suture is threaded through the clip (such as through one or more eyelets) in a manner that permits tightening of the suture. For example, the suture can be arranged in a purse-string or X pattern relative to the clip. This embodiment varies from the previous embodiments in that there is no automatic hemostasis or sheath retentions force. The sutures act as “preclose” sutures as described in the introduction, but can be applied in more limited incision areas and require less surgical skill. After the procedural sheath is removed from the clip, the pre-threaded suture is tied off, to accomplish hemostasis.

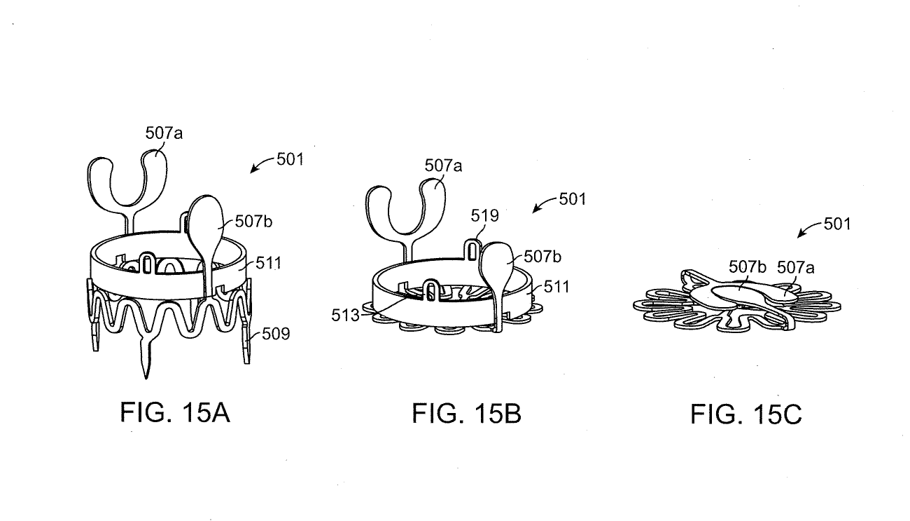

FIGS. 12A and 12B show a first embodiment of the pre-tied clip wherein a single clip 401 (formed of an annular body) has at least one tissue attachment member such as a tine for attaching to tissue and one or more sutures 403 threaded through the clip, such as through eyelets 407 in the clip member 401. FIG. 12A shows the clip member 401 in a first, untightened state such that the clip 401 is round or otherwise enlarged. A tightening force can be applied to clip 401 by pulling on the one or more sutures 403. The suture 403 exerts sufficient force to cause the clip member 401 to collapse and thereby close the arteriotomy to which it is attached, as shown in FIG. 12B

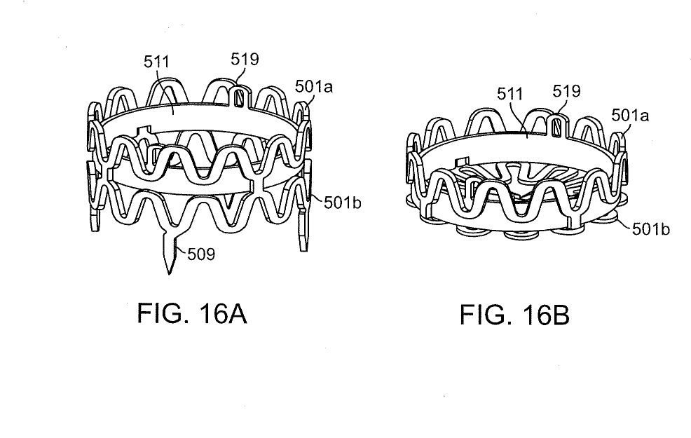

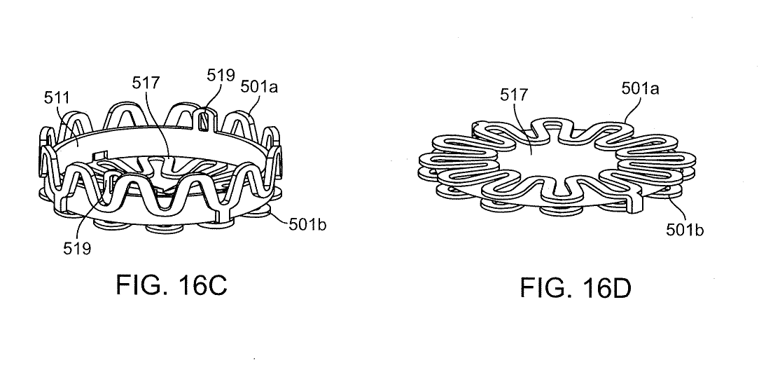

In another embodiment shown in FIGS. 13 and 14, the pre-tied clip includes a pair of clip members 401a and 401b that are attached to one another by one or more sutures 403 threaded through the clip members 401. The clip members 401a and 401b can have any of a variety of shapes including curved clip members 401 (shown in FIG. 13), straight clip members 401 (shown in FIG. 14) and/or curvilinear clip members. The suture 403 can be tightened to draw the clip members 401a and 401b toward one another so as to apply a closure force to the arteriotomy. Any quantity of clip members can be used in combination with one or more sutures.

### Combination of Springs/Clips and Sealing Material

Another embodiment of the closure device is a combination of a clip and separate seal member. The clip anchors to the vessel wall and includes features which capture the seal member over the arteriotomy after removal of the procedural sheath. The seal member may be any hemostatic material such as Dacron, collagen or other biologic matrix, bioabsorbable polymer, or other known hemostatic material.

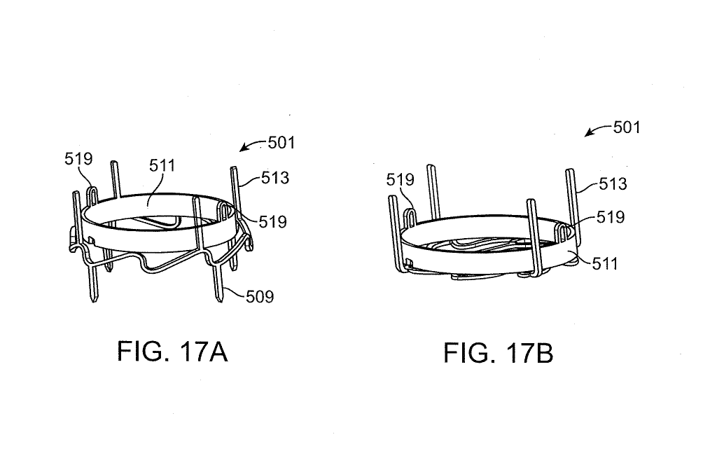

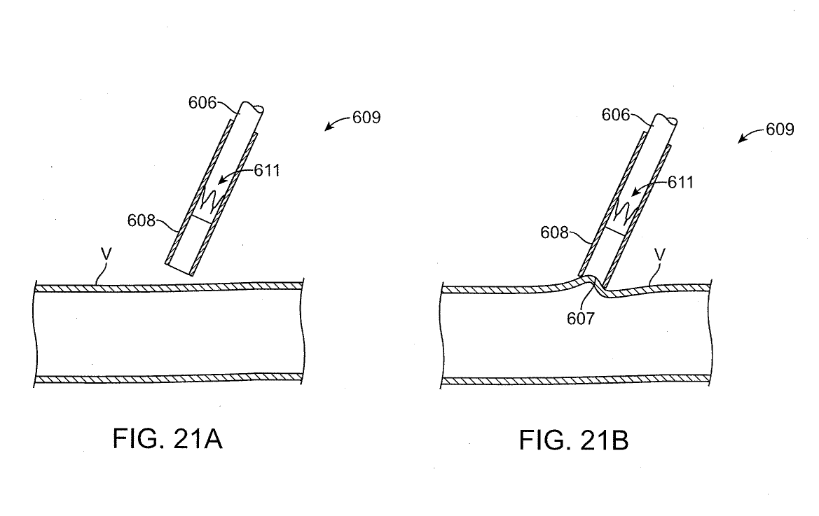

FIGS. 15A-15C show an embodiment of a combination clip 501 that combines a closure clip with a spring-loaded sealing element 507. The clip 501 may be configured the same as or similar to the clip 101 described above or the clip 501 may simply be a ring. Thus, the clip 501 may include an annular body, which may be generally annular in shape and which surrounds a central axis, and a plurality of tines 509 extending from the body. The body is configured to receive a procedural sheath that can be inserted into a blood vessel, as described more fully below. The sealing element 507 is an element that is adapted to seal with the wall of the blood vessel. The sealing element 507 can include one or more parts. In the embodiment of FIG. 20, the sealing element 507 includes a first sealing element 507a comprised of a U-shaped member that extends upwardly from the clip body. A second sealing element 507b also extends upward from the clip body and has a shape that fits within the cavity between the arms of the U-shaped first sealing element 507a.

FIG. 15A shows the clip 501 in a pre-deployed state as it would be configured during delivery over a central shaft of a delivery system. The sealing element 507 is retained in an open position such that it does not interfere with the central passageway in the clip 501, thereby permitting a procedural sheath to be positioned in the central passageway. The sealing element 507 (both the first sealing element 507a and the second sealing element 507b) is spring-loaded or otherwise biased to a position where it extends into the central passageway or opening and seals the arteriotomy as described more fully below.

With reference still to FIG. 15A, a retaining ring 511 is removably coupled to the clip 501 in a manner that interferes with the sealing element 507. That is, the retaining ring 511 prevents the sealing element 507 from moving into the central passageway of the clip and thereby retain the sealing element 507 in the pre-deployed state. This permits the delivery sheath to be passed through the clip 501 without interference from the sealing elements 507. In an embodiment, one or more tethers (not shown) are attached to eyelets 519 in the retaining ring to prevent potential loss of the ring in the body cavity during removal. The tether or tethers may also be used to remove the retaining ring.

FIG. 15B shows the clip 501 after it has deployed in the vessel wall over the arteriotomy. The annular body of the clip 501 has achieved the planar state so that it exerts a closure force onto the arteriotomy. One or more removal elements, such as tethers (not shown) can be attached to eyelets 519 on the retaining ring 511 for exerting a removal force thereon. After the delivery sheath is removed from the clip, the tethers can be pulled to disengage the retaining ring 511 from the clip 511 and the sealing elements 507. The sealing elements 507 then spring to the deployed state shown in FIG. 15C. In the deployed state, the sealing elements 507 mate with one another to seal the arteriotomy. Note that the second sealing element 507b fits within the cavity in the first sealing element 507a.

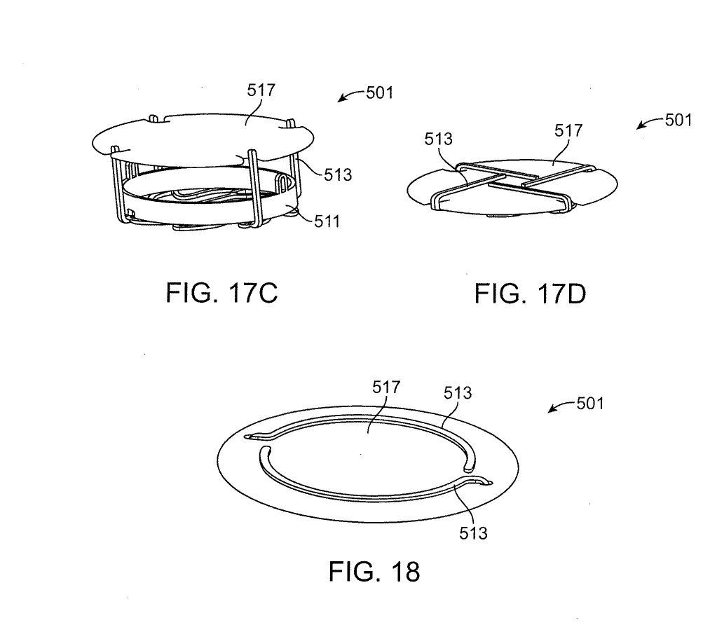

FIGS. 16A-16D show another embodiment of a clip that includes an upper clip member 501a positioned over a lower clip member 501b. As discussed below, the upper clip member acts as fastener element that fastens a seal member to the clip. Each of the clip members 501a and 501b is formed of an annular body with an undulating loop configuration in the manner described above with reference to FIG. 2. One or more tines 509 extend downward from the lower clip member 501b. FIG. 16A shows the clip in a pre-deployed state as it would be configured during delivery over a central shaft of a delivery system. A retaining ring 511 couples to the clips 501a and 501b to maintain the clips in the cylindrical or open state. In use, the bottom clip member 501b inserts into the blood vessel wall via the tines 509. The bottom clip member 501b is then permitted to collapse into the planar state, as shown in FIG. 16B. A procedural sheath can then be inserted through the center of the upper and lower clip members into the blood vessel. After the procedural sheath is removed, the retaining ring 511 maintains the upper clip member 501a in the open or cylindrical state, as shown in FIG. 16B.

With reference to FIG. 16C, a sealing member 517 can then be positioned between the lower clip member 501b and the upper clip member 501a such that the sealing member 517 is positioned over the arteriotomy. The retaining ring 511 is then removed such as by pulling on the retaining ring 511 using a tether attached to eyelets 519. The removal of the retaining ring 511 removes interference with the upper clip member 501a such that the upper clip member 501a can collapse over the sealing member 517, as shown in FIG. 16D. The upper clip member 501a and lower clip member 501b thus capture and retain the sealing member 517 in a fixed position over the arteriotomy.

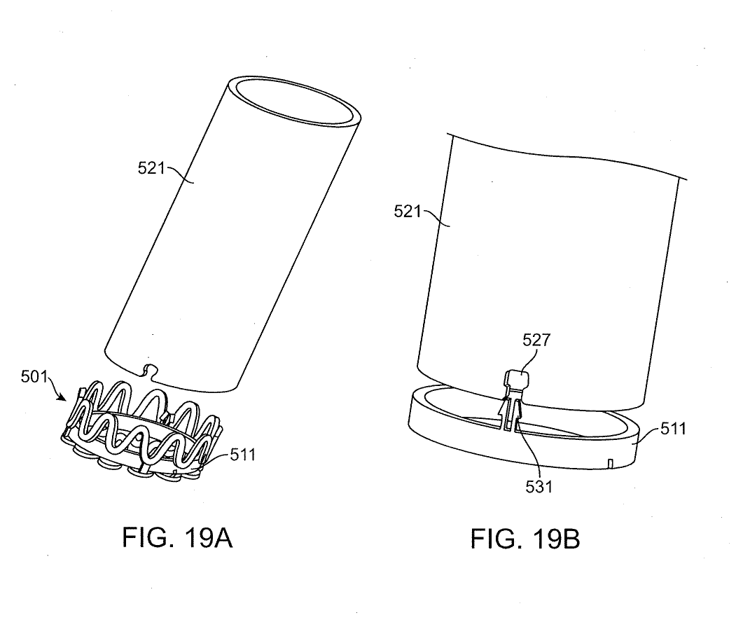

FIGS. 17A-17D shows yet another embodiment of a combination clip 501 having an annular body that includes one or more tines 509. The tines 509 insert into and attach to the blood vessel wall. The clip 501 also includes one or more upwardly extending fasteners comprised of prongs 513 that are configured to couple or fasten to a sealing element 517 (FIG. 17C) such as by inserting through holes in the sealing element 517. A retaining ring 511 interferes with and retains the prongs 513 in an open state as shown in FIG. 17A.

FIG. 17B shows the clip 501 as it is when deployed in the vessel wall so as to apply a closure force to the arteriotomy in the manner described above with reference to the clip 101. The prongs 513 are still retained in the open position by the presence of the retaining ring 511. The procedural sheath can then be inserted into and out of the clip 501. After the procedural sheath is removed, a sealing element 517 is loaded onto the prongs 513, as shown in FIG. 17C. With the sealing element 517 in place, the retaining ring 511 is then removed such as by pulling on a tether attached to eyelets 519. The prongs 513 are then allowed to transition downward into a closed state onto the sealing element 517. The prongs 513, when in the downward position or closed state, retain the sealing element 517 in place as shown in FIG. 17D.

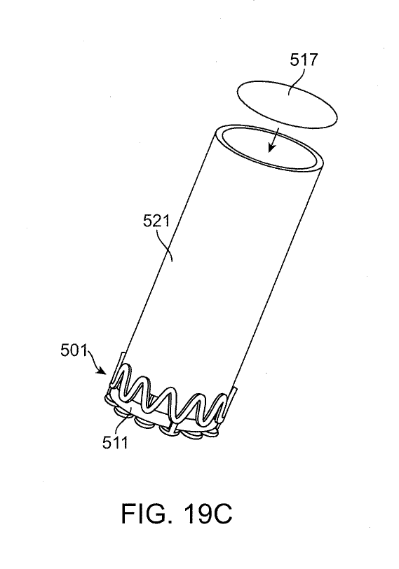

In another embodiment shown in FIG. 18, the clip member 501 includes one or more prongs 513 that have a default closed state which allows passage of the procedure sheath, such that a retaining ring is not required to maintain the prongs 513 in an open state. The clip member 501 also includes tines that attach to the blood vessel. The tines are not visible from the view of FIG. 18. The sealing member 517 is applied to the clip by lifting the prongs upward to provide a seat for the sealing member 517 over the clip. The sealing member 517 is then placed onto the clip member 501 and the prongs 513 are released so that they return to the closed state and retain the sealing member 517 in place.

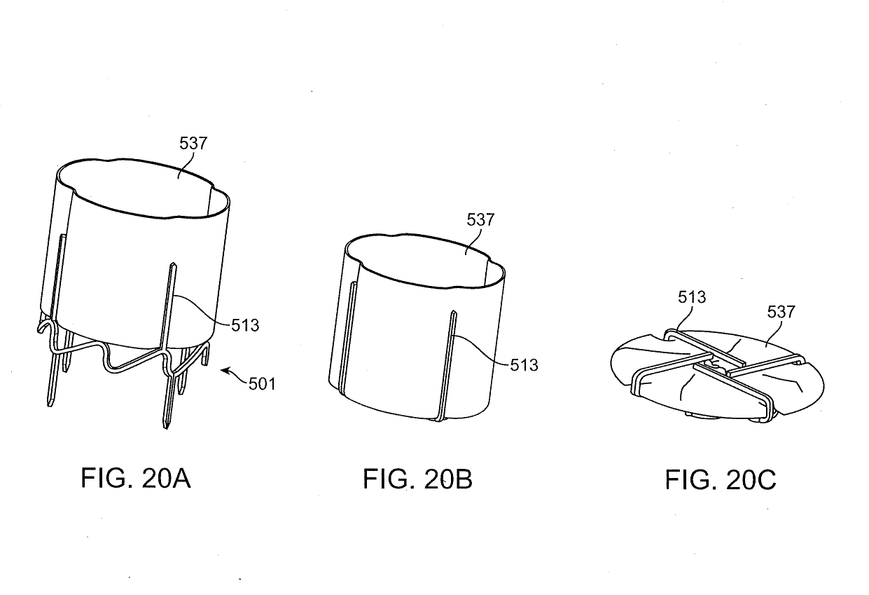

FIGS. 19A-19C show an exemplary device for removing the retaining ring from any of the clip embodiments with retaining rings. In this embodiment, a removal member comprises an elongate tube 521 having a lower end that attaches to the retaining ring 511. As shown in the enlarged view of FIG. 19B, the lower end of the tube 521 has one or more features, such as notches 527, that attach to one or more features, such as protrusions 531, on the retaining ring 511. As the tube 521 is lowered toward the retaining ring 511, the protrusions 531 insert into the notches 527 in a manner that couples the tube 521 to the retaining ring 511. In embodiments with a separate seal material as in FIGS. 16, 17, and 18, the tube 521 can also be used to guide the seal member 517 in place, as shown in FIG. 19C. In this case, the seal member 517 may be pushed down with a push rod. While the rod is holding the seal material in place, the tube 521 can then be lifted off the clip to remove the attached retaining ring 511. Alternately, the tube itself can serve as the retaining ring. The tube 521 then remains in place during the entire procedure. As before, the tube can then be used to guide the seal material in place before being removed.

The tube 521 can also be pre-loaded onto the procedural sheath so it may slide down over the procedural sheath before the procedural sheath is removed. In this way, the tube 521 can act as a counter traction against the clip 501 while the procedural sheath is being removed.

In another embodiment shown in FIGS. 20A-20C, the sealing member is a cylindrical sealing sleeve 537 that is preattached to the clip 501. The sleeve has a height such that a set of prongs 513 can be positioned over the sleeve 537 to retain it in place during deployment of the clip 501, as shown in FIG. 20A. After the clip 501 is deployed in the blood vessel, the prongs 513 and sealing sleeve 537 are initially maintained in an open state as shown in FIG. 20B. The prongs 513 are then permitted to collapse inward and retain the sealing sleeve 537 in place as shown in FIG. 20C.

### Delivery of Clip

Various features and modalities can be employed to deliver the clip onto the blood vessel and arteriotomy. A delivery system can be coupled to the clip and used to deliver the clip onto the blood vessel. The delivery system may include a delivery device comprising a central delivery shaft such as a cylindrical member over which the clip is mounted. A retaining sleeve is positioned coaxially over the central delivery shaft and clip and prevents the clip from expanding outward and/or slipping from the central delivery shaft during delivery. A vessel locator may be included to assist in locating the distal tip of the delivery system securely against the vessel wall. A proximal actuator may push the clip from the central delivery shaft and retract the retaining sleeve to deploy the clip into the vessel wall. The delivery system may also include a central guidewire lumen (such as through the central delivery shaft) and be delivered to the outer surface of the vessel over a guidewire pre-positioned in the vessel. The guidewire may then remain in place while the delivery system is removed and then be used to delivery the procedural sheath through the deployed clip. Alternately, the delivery system may incorporate the procedural sheath as the central delivery shaft of the delivery system. In another embodiment, the central delivery shaft and procedural sheath are two separate components that are integrated into a single delivery system. In these embodiments, the clip delivery shaft and procedural sheath combination systems may also be delivered over a guidewire.

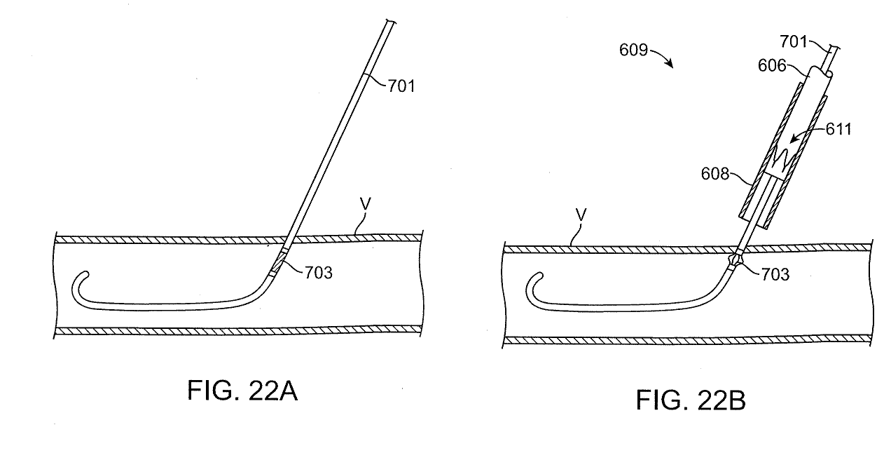

In one embodiment, suction is used in combination with the delivery system during delivery of the clip. Various configurations can be used to apply suction, such as a syringe, suction cartridge, suction pump, wall suction, etc. The suction functions to secure at least a portion of the delivery system to the exterior surface of the vessel wall for reliable clip delivery to the vessel wall. FIG. 21A shows a suction delivery system 609 that is used to deliver the clip 611 (which can be any of the clip embodiments described herein or any type of closure clip not limited to the clips described herein) to a blood vessel V. The clip 611 is mounted on a central delivery shaft 606 that is positioned coaxially within a retaining sleeve 608. As shown in FIG. 21B, a suction force can be applied to the vessel wall via the delivery system 609. The delivery system 609 applies suction via an internal lumen in a component of the delivery system 609 such that the suction force gather a region 607 of tissue into a portion of the delivery system 609, such as the retaining sleeve 608. With the region 607 gathered into the retaining sleeve 608, the clip 611 can more easily latch onto the tissue. The gathered tissue also creates the ability to create a bigger “bite” for closure, in other words, a greater distance between attachment points, thus potentially improving the security and closure force of the clip device.

The delivery system may include a clip carrier assembly having an elongated member that retains the vessel closure clip in a delivereable configuration during clip delivery. The carrier assembly is adapted to deploy the vessel closure clip onto the artery. The carrier assembly may include an actuation element that actuates a pusher member with respect to an elongated member to push the clip off the elongated member and deploy the clip. The carrier assembly may further comprise a cover member for retaining the vessel closure clip on the elongated member during delivery.

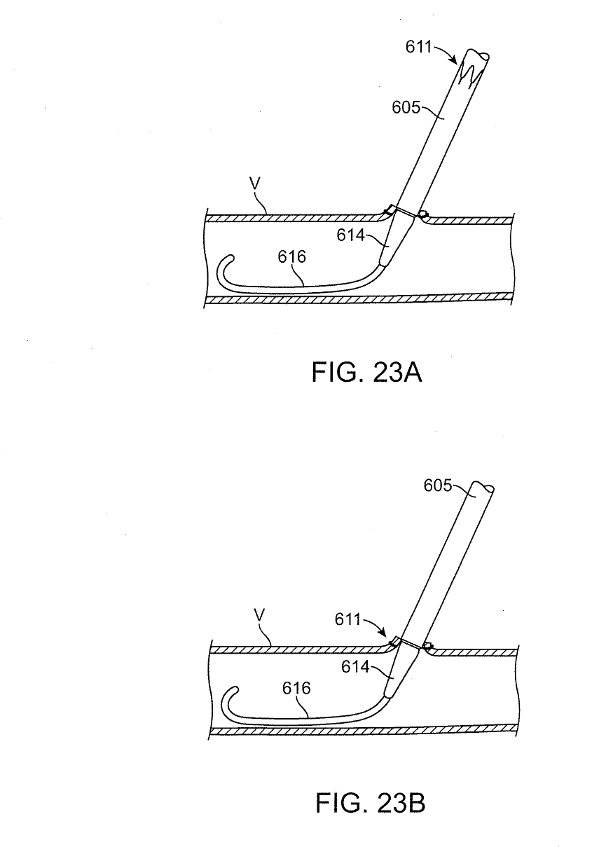

In another embodiment, a locating member in the form of a guidewire or small mandrel can be employed to position the delivery system with respect to the vessel wall during clip delivery. FIG. 22A shows a locating device 701 in the form of a guidewire having an expandable vessel wall locator 703 positioned thereon. The locating device 701 is first inserted into the vessel with the vessel wall locator 703 in a collapsed, generally mandrel state, as shown in FIG. 22A. As shown in FIG. 22B, the vessel wall locator 703 is then expanded, for example by an actuator (not shown) on the proximal end of the locating device 701. The vessel wall locator 703 is then positioned against the vessel wall from inside the vessel. The clip delivery system 609 (including the central delivery shaft 606 and the retaining sleeve 608) is guided to the vessel wall over the locating device 701 and the clip 611 is deployed. If a guidewire form is used, it may remain in place after the clip 611 is deployed and the delivery device is removed, and then be used to deliver the procedural sheath. Suction can be applied in combination with the vessel locating device 701.

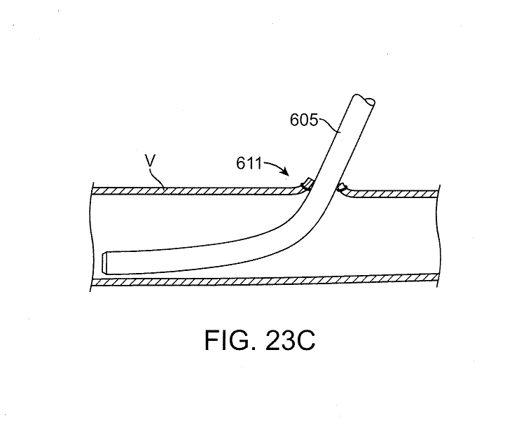

In yet another embodiment, shown in FIGS. 23A-23C, the clip 611 may be pre-mounted on the procedural sheath 605 such that the procedural sheath 605 serves as the central delivery shaft of the delivery system. In this case, as shown in FIG. 23A, the procedural sheath 605 is inserted through the penetration in the vessel and into the vessel V via conventional means such as a micropuncture technique or modified Seldinger technique. The procedural sheath 605 may be coupled to a dilator 614 and a guidewire 616. The pre-mounted clip 611 is then pushed over the procedural sheath 605 toward the blood vessel V. The clip 611 is deployed around the vessel at the site of procedural sheath insertion, as shown in FIG. 23B. After the clip 611 is deployed, the guidewire 616 and dilator 614 may be removed, as shown in FIG. 23C, while the procedural sheath 605 stays in place to provide access for a procedural device that may be inserted through the procedural sheath 605 into the blood vessel V for performing a procedure. As with previous embodiments, after procedural sheath removal the clip then closes the arteriotomy.

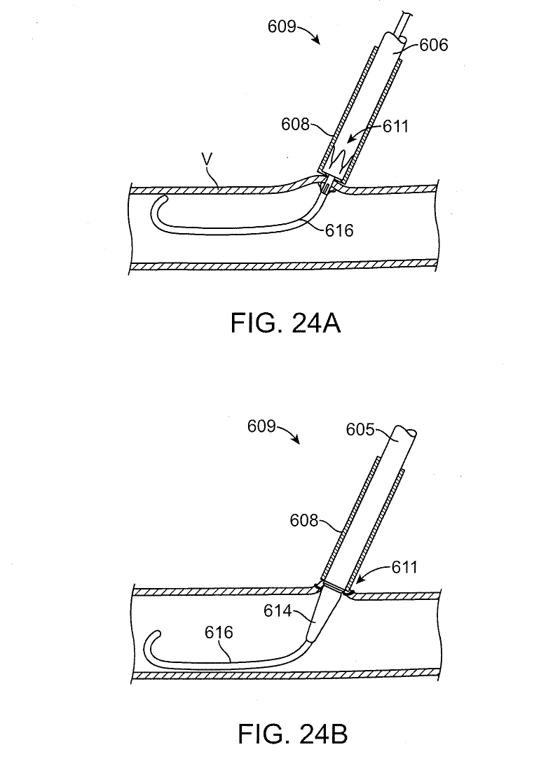

In yet another embodiment, shown in FIGS. 24A-24C, the procedural sheath 605 may be mounted on the central delivery shaft 606 of the delivery system 609. The procedural sheath 605 may be pre-mounted on a proximal region of the central delivery shaft 606 such that the procedural sheath 605 can slide distally over the delivery shaft 606 and through the central opening of the clip 611. The procedural sheath 605 may have a hemostasis valve, such as on the proximal end of the procedural sheath. Thus, when the delivery system 609 is removed, hemostasis is maintained. If a procedural sheath 605 is used which requires a proximal extended section (as described below), an attachable extension can be added to the proximal end of the procedural sheath 605 after removal of the clip delivery system 609. Alternately, the delivery shaft 606 can have an extended length that permits pre-mounting of both the procedural sheath and proximal extension. In another embodiment, the procedural sheath 605 is not pre-mounted on the central delivery shaft 606 but is exchanged with the central delivery shaft 605 in conjunction with or after removal of the delivery shaft 606 from the blood vessel V. After the clip 611 is delivered, the procedural sheath 605 may be advanced through the retaining sleeve 608 of the delivery system 609 and through the clip 611 into the blood vessel V, as shown in FIG. 24B. The procedural sheath 605 may be coupled to a dilator 614 during this process. The delivery shaft 606, retaining sheath 608, dilator 614, and guidewire 616 (if present) are then removed, leaving the procedural sheath 605 and clip 611 in place, as shown in FIG. 24C. The procedural sheath 605 stays in place to provide access for a procedural device that may be inserted through the procedural sheath 605 into the blood vessel V for performing a procedure. At the end of the procedure, the procedural sheath 605 is removed, and the clip 611 seals the vessel opening.

The procedural sheath 605 may include an intravascular occlusion element for procedures requiring arterial occlusion. The intravascular occlusion element may be, for example, an inflatable balloon, an expandable member such as a braid, cage, or slotted tube around which is a sealing membrane, or the like. The procedural sheath may also include a sheath retention element such as an inflatable structure or an expandable wire, cage, or articulating structure which prevents inadvertent sheath removal from the blood vessel when the sheath is deployed.

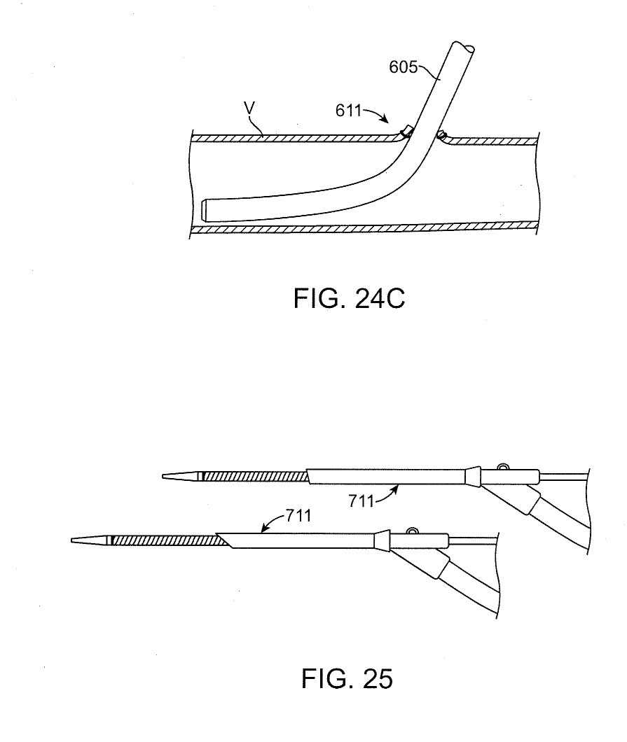

The delivery device can include a countertraction feature that prevents the clip from being detached from the blood vessel during removal of the delivery device. Similarly, the procedural sheath can include a counter traction feature that prevents the clip from being detached during removal of the sheath. For example, as shown in FIG. 25, a tube 711 can be located on the outside of the sheath. The tube 711 acts as a countertraction feature and is pushed forward along the outer surface of the procedural during sheath removal. The distal tip of the countertraction tube 711 abuts the clip and is held against the vessel wall to hold the preclose clip in place and prevent inadvertent removal of the clip during sheath removal. The distal tip of the tube 711 can be shaped in various manners depending on which pre-close clip embodiment is being used. For example, the tip may be blunt or beveled, to act as “sheath stop” to prevent the sheath from entering vessel too far. The tube 711 can have an extended tip that goes through clip so that clip does not interfere with sheath removal.

### Description of Exemplary Retrograde Flow System

Any of the embodiments of the closure clips discussed above may be used in combination with a retrograde flow system that may be used in conjunction with a variety of interventional procedures. It should be appreciated that the retrograde flow system can also be used in combination with other types of closure devices different than those described herein. Exemplary embodiments of a retrograde flow system and exemplary interventional procedures are now described.

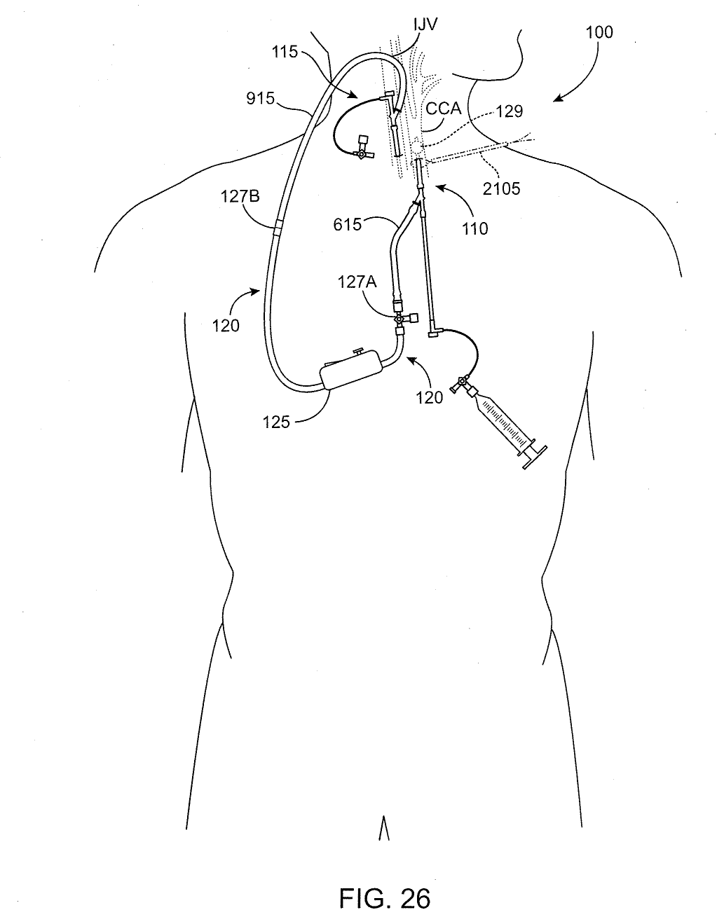

FIG. 26 shows an exemplary embodiment of a retrograde flow system 100 that is adapted to establish and facilitate retrograde or reverse flow blood circulation, such as in the region of the carotid artery bifurcation in order to limit or prevent the release of emboli into the cerebral vasculature, particularly into the internal carotid artery. Although described in the context of being used in the region of the carotid artery bifurcation to limit or prevent the release of emboli into the cerebral vasculature, it should be appreciated that the system can be used in accordance with various neurointerventional procedures in various anatomical locations.

In an embodiment, the system 100 interacts with the carotid artery to provide retrograde flow from the carotid artery to a venous return site, such as the internal jugular vein (or to another return site such as another large vein or an external receptacle in alternate embodiments.) The retrograde flow system 100 includes an arterial access device 110, a venous return device 115, and a shunt 120 that provides a passageway for retrograde flow from the arterial access device 110 to the venous return device 115. A flow control assembly 125 interacts with the shunt 120. The flow control assembly 125 is adapted to regulate and/or monitor the retrograde flow from the common carotid artery to the internal jugular vein, as described in more detail below. The flow control assembly 125 interacts with the flow pathway through the shunt 120, either external to the flow path, inside the flow path, or both.

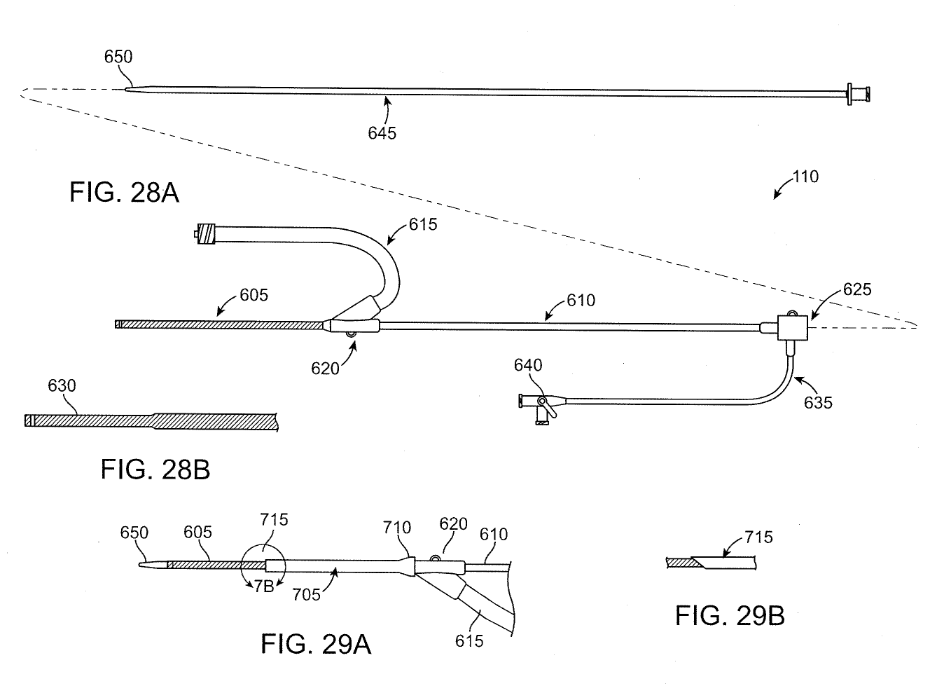

The arterial access device 110 at least partially inserts into the common carotid artery CCA. In this regard, the arterial access device 110 includes a procedural sheath 605 (described below with reference to FIG. 28A-30B). The procedural sheath 605 can interface with any of the embodiments of the clip closure devices described herein to close the access way into the blood vessel. Thus, any of the clips can be premounted on the sheath 605.

The venous return device 115 at least partially inserts into a venous return site such as the internal jugular vein IJV, as described in more detail below. The arterial access device 110 and the venous return device 115 couple to the shunt 120 at connection locations 127a and 127b. When flow through the common carotid artery is blocked, the natural pressure gradient between the internal carotid artery and the venous system causes blood to flow in a retrograde or reverse direction from the cerebral vasculature through the internal carotid artery and through the shunt 120 into the venous system. The flow control assembly 125 modulates, augments, assists, monitors, and/or otherwise regulates the retrograde blood flow.

In the embodiment of FIG. 26, the arterial access device 110 accesses the common carotid artery CCA via a transcervical approach. Transcervical access provides a short length and non-tortuous pathway from the vascular access point to the target treatment site thereby easing the time and difficulty of the procedure, compared for example to a transfemoral approach. Additionally, this access route reduces the risk of emboli generation from navigation of diseased, angulated, or tortuous aortic arch or common carotid artery anatomy. At least a portion of the venous return device 115 is placed in the internal jugular vein IJV. In an embodiment, transcervical access to the common carotid artery is achieved percutaneously via an incision or puncture in the skin through which the arterial access device 110 is inserted. If an incision is used, then the incision can be about 0.5 cm in length. As mentioned, any of the closure clips described herein can be used to close the incision.

An occlusion element 129, such as an expandable balloon, can be used to occlude the common carotid artery CCA at a location proximal of the distal end of the arterial access device 110. The occlusion element 129 can be located on the arterial access device 110 or it can be located on a separate device. In an alternate embodiment, the arterial access device 110 accesses the common carotid artery CCA via a direct surgical transcervical approach. In the surgical approach, the common carotid artery can be occluded using a tourniquet 2105.

In another embodiment, the arterial access device 110 accesses the common carotid artery CCA via a transcervical approach while the venous return device 115 access a venous return site other than the jugular vein, such as a venous return site comprised of the femoral vein. The venous return device 115 can be inserted into a central vein such as the femoral vein FV via a percutaneous puncture in the groin.

In another embodiment, the arterial access device 110 accesses the common carotid artery via a femoral approach. According to the femoral approach, the arterial access device 110 approaches the CCA via a percutaneous puncture into the femoral artery FA, such as in the groin, and up the aortic arch into the target common carotid artery CCA. The venous return device 115 can communicate with the jugular vein or the femoral vein.

In another embodiment, the system provides retrograde flow from the carotid artery to an external receptacle 130 rather than to a venous return site. The arterial access device 110 connects to the receptacle 130 via the shunt 120, which communicates with the flow control assembly 125. The retrograde flow of blood is collected in the receptacle 130. If desired, the blood could be filtered and subsequently returned to the patient. The pressure of the receptacle 130 could be set at zero pressure (atmospheric pressure) or even lower, causing the blood to flow in a reverse direction from the cerebral vasculature to the receptacle 130. Optionally, to achieve or enhance reverse flow from the internal carotid artery, flow from the external carotid artery can be blocked, typically by deploying a balloon or other occlusion element in the external carotid artery just above the bifurcation with the internal carotid artery.

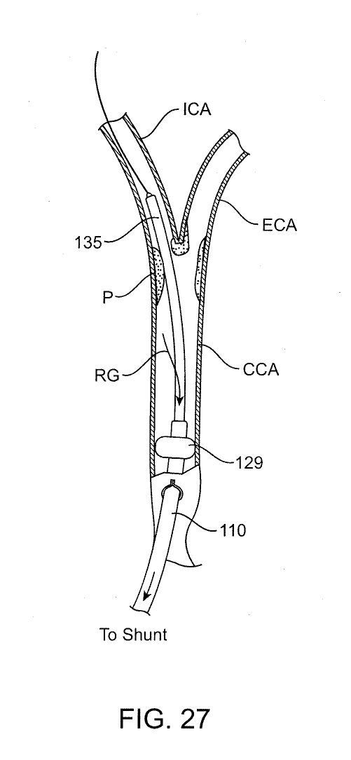

With reference to the enlarged view of the carotid artery in FIG. 27, an exemplary interventional device (which may also referred to as a procedural device), such as a stent delivery system 135 including a stent delivery catheter or other working catheter, can be introduced into the carotid artery via the arterial access device 110, as described in detail below. The stent delivery system 135 can be used to treat the plaque P such as to deploy a stent into t a carotid or cerebral artery. The arrow RG in FIG. 27 represents the direction of retrograde flow. As mentioned, a stent delivery interventional device and method is just an example of an intervention that can be used in conjunction with the clip closure devices described herein. Other interventions are possible such as, for example, intracerebral balloon angioplasty, an acute ischemic stroke treatment procedure, treatment of intracerebral aneurysms, arteriovenous malformations, or other intracerebral procedures.

In an embodiment, the system and closure elements are used in accordance with a procedure involving the introduction into an aneurysm of a solid endovascular implant such as a coil or braid and a polymeric composition which may be reformed or solidified in situ for stabilizing and at least partially filling the aneurysm. The solid endovascular implant is at least partially surrounded or enveloped by the polymeric composition. The polymeric composition is reformed via light, heat, R.F. or the like to form a rigid mass with the solid endovascular implant. These steps may be carried out sequentially or the steps of introducing the endovascular implant and reforming the polymeric composition may be carried out simultaneously. The procedure may be accomplished using an intravascular catheter similar to the catheter to access the desired site and to deliver the noted materials.

In another embodiment, the interventional device is an embolic system which can deliver an embolic material or fluid composition through a microcatheter into the blood vessel. The material or composition solidifies and/or expands to fully or partially occlude a vascular site. The term “embolizing” or “embolization” refers to a process wherein a material or fluid composition is injected into a blood vessel which, in the case of, for example, aneurysms, fills or plugs the aneurysm sac and/or encourages clot formation so that blood flow into the aneurysm and pressure in the aneurysm ceases, and in the case of arterial venous malformations (AVMs) and arterial venous fistula (AVFs) forms a plug or clot to control/reroute blood flow to permit proper tissue perfusion. Embolization may be used for preventing/controlling bleeding due to lesions (e.g., organ bleeding, gastrointestinal bleeding, vascular bleeding, as well as bleeding associated with an aneurysm). In addition, embolization can be used to ablate diseased tissue (e.g., tumors, etc.) by cutting off its blood supply. U.S. Pat. Nos. 6,146,373 and 5,443,454 (which are both are incorporated herein by reference) describe exemplary liquid embolic systems.

In another embodiment, the interventional device is a microcatheter used to delivery therapeutic agents such as cerebral protective agents, chemotherapeutic agents, stem cell or other regenerative agents, neurochemical or neuropsychopharmacologic agents, or the like, to an intracranial or cerebral artery and/or the brain.

In yet another embodiment, the interventional device is a balloon dilatation or balloon occlusion catheter. In yet another embodiment, the interventional device 15 is a thrombus disruption or removal system. In yet another embodiment, the interventional device is a brain tumor treatment device or a diagnostic angiography catheter.

### Detailed Description of Retrograde Blood Flow System

As discussed, the retrograde flow system 100 includes the arterial access device 110, venous return device 115, and shunt 120 which provides a passageway for retrograde flow from the arterial access device 110 to the venous return device 115. The system also includes the flow control assembly 125, which interacts with the shunt 120 to regulate and/or monitor retrograde blood flow through the shunt 120. Exemplary embodiments of the components of the retrograde flow system 100 are now described.

Arterial Access Device

FIG. 28A shows an exemplary embodiment of the arterial access device 110, which comprises a distal sheath 605, a proximal extension 610, a flow line 615, an adaptor or Y-connector 620, and a hemostasis valve 625. The distal sheath 605 is adapted to be introduced through an incision or puncture in a wall of a common carotid artery, either an open surgical incision or a percutaneous puncture established, for example, using the Seldinger technique. The length of the sheath can be in the range from 5 to 15 cm, usually being from 10 cm to 12 cm. The inner diameter is typically in the range from 7 Fr (1 Fr=0.33 mm), to 10 Fr, usually being 8 Fr. Particularly when the sheath is being introduced through the transcervical approach, above the clavicle but below the carotid bifurcation, it is desirable that the sheath 605 be highly flexible while retaining hoop strength to resist kinking and buckling. Thus, the distal sheath 605 can be circumferentially reinforced, such as by braid, helical ribbon, helical wire, or the like. In an alternate embodiment, the distal sheath is adapted to be introduced through a percutaneous puncture into the femoral artery, such as in the groin, and up the aortic arch AA into the target common carotid artery CCA

The distal sheath 605 can have a stepped or other configuration having a reduced diameter distal region 630, as shown in FIG. 28B, which shows an enlarged view of the distal region 630 of the sheath 605. The distal region 630 of the sheath can be sized for insertion into the carotid artery, typically having an inner diameter in the range from 2.16 mm (0.085 inch) to 2.92 mm (0.115 inch) with the remaining proximal region of the sheath having larger outside and luminal diameters, with the inner diameter typically being in the range from 2.794 mm (0.110 inch) to 3.43 mm (0.135 inch). The larger luminal diameter of the proximal region minimizes the overall flow resistance of the sheath. In an embodiment, the reduced-diameter distal section 630 has a length of approximately 2 cm to 4 cm. The relatively short length of the reduced-diameter distal section 630 permits this section to be positioned in the common carotid artery CCA via the transcervical approach with reduced risk that the distal end of the sheath 605 will contact the bifurcation B. Moreover, the reduced diameter section 630 also permits a reduction in size of the arteriotomy for introducing the sheath 605 into the artery while having a minimal impact in the level of flow resistance.

With reference again to FIG. 28A, the proximal extension 610 has an inner lumen which is contiguous with an inner lumen of the sheath 605. The lumens can be joined by the Y-connector 620 which also connects a lumen of the flow line 615 to the sheath. In the assembled system, the flow line 615 connects to and forms a first leg of the retrograde shunt 120. The proximal extension 610 can have a length sufficient to space the hemostasis valve 625 well away from the Y-connector 620, which is adjacent to the percutaneous or surgical insertion site. By spacing the hemostasis valve 625 away from a percutaneous insertion site, the physician can introduce a stent delivery system or other working catheter into the proximal extension 610 and sheath 605 while staying out of the fluoroscopic field when fluoroscopy is being performed.

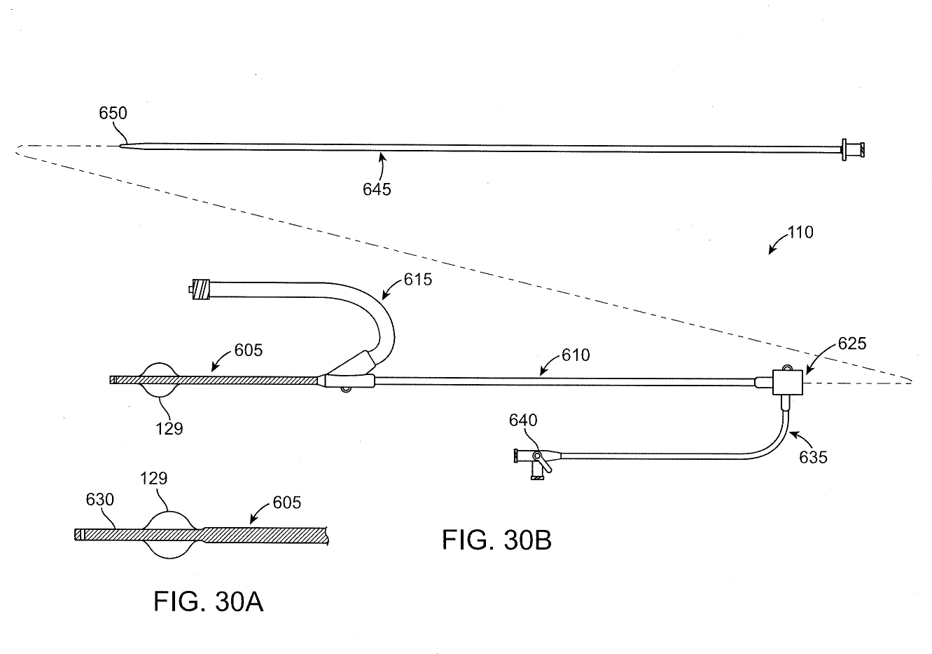

A flush line 635 can be connected to the side of the hemostasis valve 625 and can have a stopcock 640 at its proximal or remote end. The flush-line 635 allows for the introduction of saline, contrast fluid, or the like, during the procedures. The flush line 635 can also allow pressure monitoring during the procedure. A dilator 645 having a tapered distal end 650 can be provided to facilitate introduction of the distal sheath 605 into the common carotid artery. The dilator 645 can be introduced through the hemostasis valve 625 so that the tapered distal end 650 extends through the distal end of the sheath 605, as best seen in FIG. 29A. The dilator 645 can have a central lumen to accommodate a guide wire. Typically, the guide wire is placed first into the vessel, and the dilator/sheath combination travels over the guide wire as it is being introduced into the vessel.

Optionally, a tube 705 may be provided which is coaxially received over the exterior of the distal sheath 605, also as seen in FIG. 29A. The tube 705 has a flared proximal end 710 which engages the adapter 620 and a distal end 715. Optionally, the distal end 715 may be beveled, as shown in FIG. 29B. The tube 705 may serve at least two purposes. First, the length of the tube 705 limits the introduction of the sheath 605 to the exposed distal portion of the sheath 605, as seen in FIG. 29A. Second, the tube 705 can engage a pre-deployed puncture closure device disposed in the carotid artery wall, if present, to permit the sheath 605 to be withdrawn without dislodging the closure device.

The distal sheath 605 can be configured to establish a curved transition from a generally anterior-posterior approach over the common carotid artery to a generally axial luminal direction within the common carotid artery. The transition in direction is particularly useful when a percutaneous access is provided through the common carotid wall. While an open surgical access may allow for some distance in which to angle a straight sheath into the lumen of the common carotid artery, percutaneous access will generally be in a normal or perpendicular direction relative to the access of the lumen, and in such cases, a sheath that can flex or turn at an angle will find great use.

In an embodiment, the sheath 605 includes a retention feature that is adapted to retain the sheath within a blood vessel (such as the common carotid artery) into which the sheath 605 has been inserted. The retention features reduces the likelihood that the sheath 605 will be inadvertently pulled out of the blood vessel. In this regard, the retention feature interacts with the blood vessel to resist and/or eliminate undesired pull-out. In addition, the retention feature may also include additional elements that interact with the vessel wall to prevent the sheath from entering too far into the vessel. The retention feature may also include sealing elements which help seal the sheath against arterial blood pressure at the puncture site.

The sheath 605 can be formed in a variety of ways. For example, the sheath 605 can be pre-shaped to have a curve or an angle some set distance from the tip, typically 2 to 3 cm. The pre-shaped curve or angle can typically provide for a turn in the range from 20° to 90°, preferably from 30° to 70°. For initial introduction, the sheath 605 can be straightened with an obturator or other straight or shaped instrument such as the dilator 645 placed into its lumen. After the sheath 605 has been at least partially introduced through the percutaneous or other arterial wall penetration, the obturator can be withdrawn to allow the sheath 605 to reassume its pre-shaped configuration into the arterial lumen.

Other sheath configurations include having a deflection mechanism such that the sheath can be placed and the catheter can be deflected in situ to the desired deployment angle. In still other configurations, the catheter has a non-rigid configuration when placed into the lumen of the common carotid artery. Once in place, a pull wire or other stiffening mechanism can be deployed in order to shape and stiffen the sheath into its desired configuration. One particular example of such a mechanism is commonly known as “shape-lock” mechanisms as well described in medical and patent literature.

Another sheath configuration comprises a curved dilator inserted into a straight but flexible sheath, so that the dilator and sheath are curved during insertion. The sheath is flexible enough to conform to the anatomy after dilator removal.

In an embodiment, the sheath has built-in puncturing capability and atraumatic tip analogous to a guide wire tip. This eliminates the need for needle and wire exchange currently used for arterial access according to the micropuncture technique, and can thus save time, reduce blood loss, and require less surgeon skill.

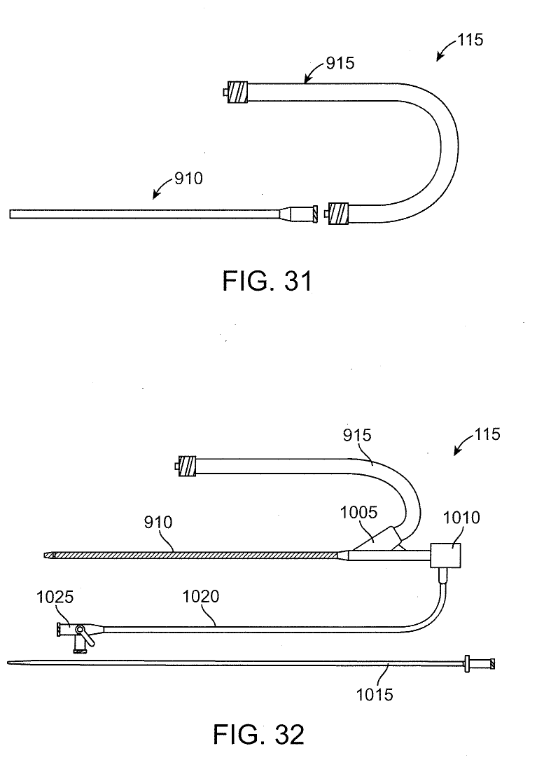

FIG. 30A shows another embodiment of the arterial access device 110. This embodiment is substantially the same as the embodiment shown in FIG. 28A, except that the distal sheath 605 includes an occlusion element 129 for occluding flow through, for example the common carotid artery. If the occluding element 129 is an inflatable structure such as a balloon or the like, the sheath 605 can include an inflation lumen that communicates with the occlusion element 129. The occlusion element 129 can be an inflatable balloon, but it could also be an inflatable cuff, a conical or other circumferential element which flares outwardly to engage the interior wall of the common carotid artery to block flow therepast, a membrane-covered braid, a slotted tube that radially enlarges when axially compressed, or similar structure which can be deployed by mechanical means, or the like. In the case of balloon occlusion, the balloon can be compliant, non-compliant, elastomeric, reinforced, or have a variety of other characteristics. In an embodiment, the balloon is an elastomeric balloon which is closely received over the exterior of the distal end of the sheath prior to inflation. When inflated, the elastomeric balloon can expand and conform to the inner wall of the common carotid artery. In an embodiment, the elastomeric balloon is able to expand to a diameter at least twice that of the non-deployed configuration, frequently being able to be deployed to a diameter at least three times that of the undeployed configuration, more preferably being at least four times that of the undeployed configuration, or larger.

As shown in FIG. 30B, the distal sheath 605 with the occlusion element 129 can have a stepped or other configuration having a reduced diameter distal region 630. The distal region 630 can be sized for insertion into the carotid artery with the remaining proximal region of the sheath 605 having larger outside and luminal diameters, with the inner diameter typically being in the range from 2.794 mm (0.110 inch) to 3.43 mm (0.135 inch). The larger luminal diameter of the proximal region minimizes the overall flow resistance of the sheath. In an embodiment, the reduced-diameter distal section 630 has a length of approximately 2 cm to 4 cm. The relatively short length of the reduced-diameter distal section 630 permits this section to be positioned in the common carotid artery CCA via the transcervical approach with reduced risk that the distal end of the sheath 605 will contact the bifurcation B.

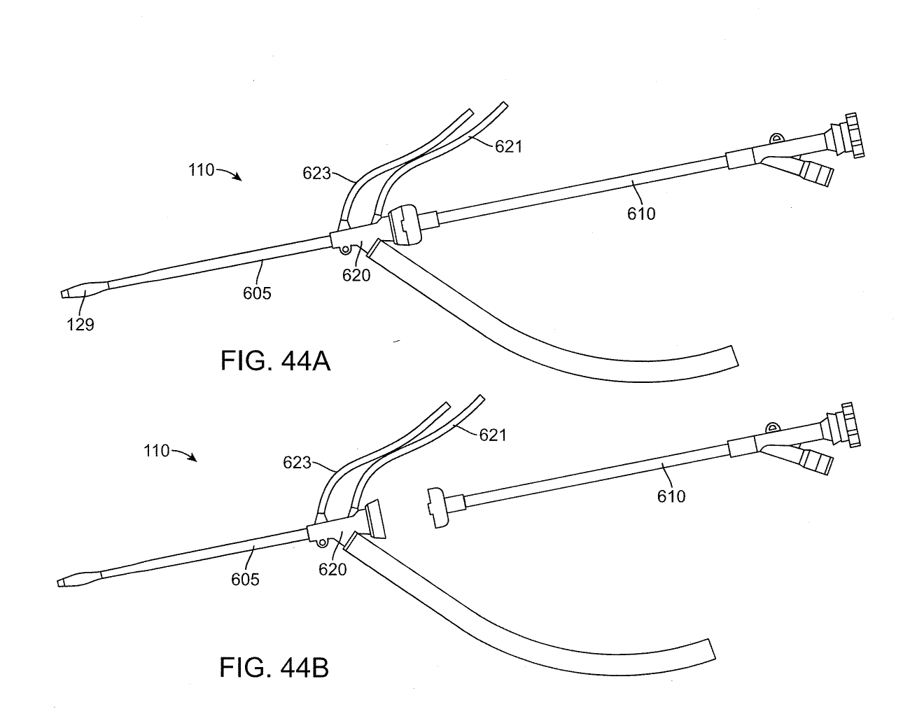

In an embodiment as shown in FIGS. 44A and 44B, the proximal extension 610 may be removably connected to the Y-arm connector 620 at a connection site. In this embodiment, an additional hemostasis valve 621 may be included at the connection site of the proximal extension 610 to the Y-arm connector 620, so that hemostasis is maintained when the proximal extension is not attached. FIG. 44A shows the arterial access sheath 605, with the proximal extension 610 attached to the Y-connector 620. FIG. 44A also shows an additional connection line 623 for balloon inflation of an occlusion element 129. FIG. 44B shows the proximal extension 610 removed from the Y-connector 620.

Venous Return Device

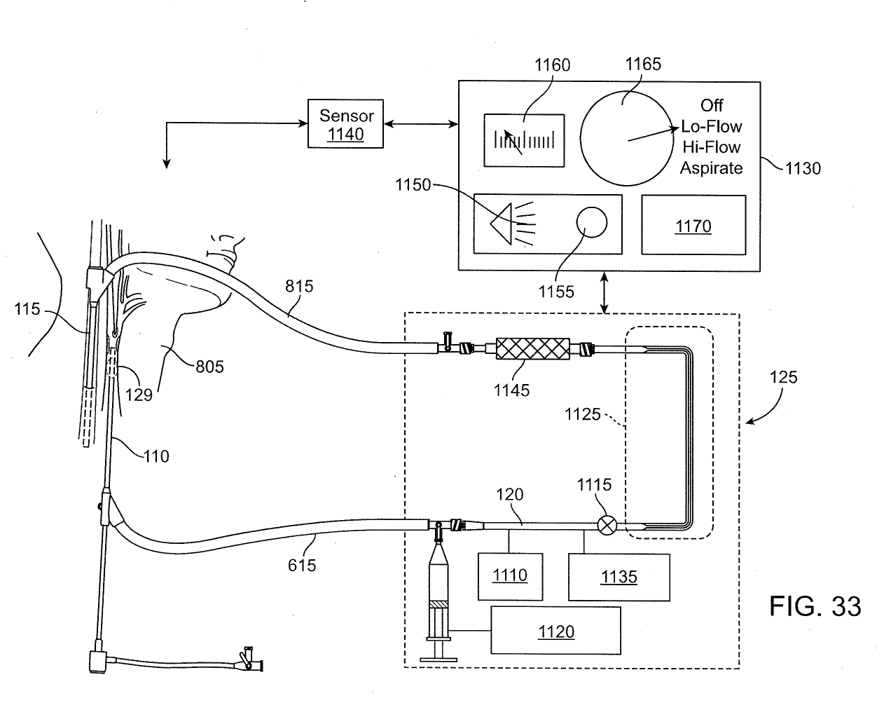

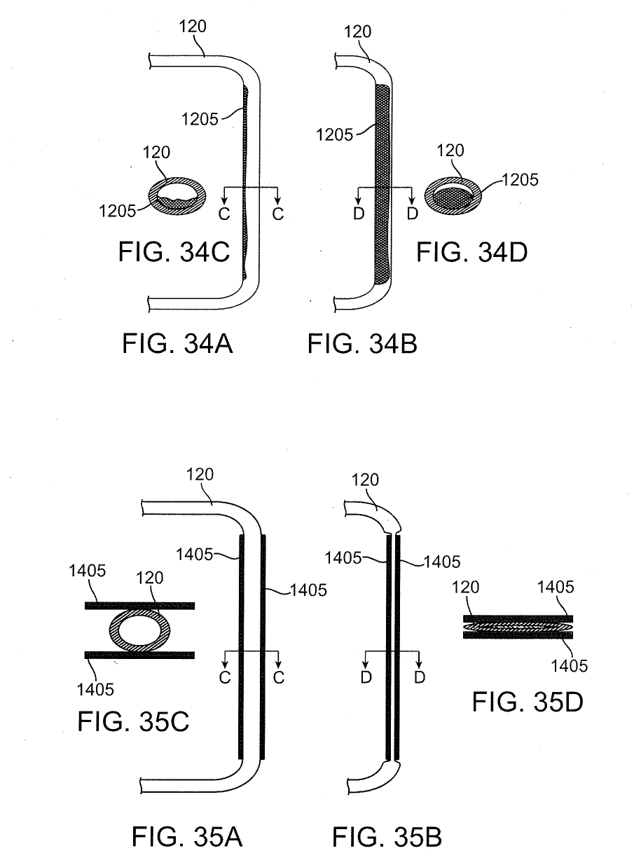

Referring now to FIG. 31, the venous return device 115 can comprise a distal sheath 910 and a flow line 915, which connects to and forms a leg of the shunt 120 when the system is in use. The distal sheath 910 is adapted to be introduced through an incision or puncture into a venous return location, such as the jugular vein or femoral vein. The distal sheath 910 and flow line 915 can be permanently affixed, or can be attached using a conventional luer fitting, as shown in FIG. 31. Optionally, as shown in FIG. 32, the sheath 910 can be joined to the flow line 915 by a Y-connector 1005. The Y-connector 1005 can include a hemostasis valve 1010, permitting insertion of a dilator 1015 to facilitate introduction of the venous return device into the internal jugular vein or other vein. As with the arterial access dilator 645, the venous dilator 1015 includes a central guide wire lumen so the venous sheath and dilator combination can be placed over a guide wire. Optionally, the venous sheath 910 can include a flush line 1020 with a stopcock 1025 at its proximal or remote end.

In order to reduce the overall system flow resistance, the arterial access flow line 615 (FIG. 28A) and the venous return flow line 915, and Y-connectors 620 (FIG. 28A) and 1005, can each have a relatively large flow lumen inner diameter, typically being in the range from 2.54 mm (0.100 inch) to 5.08 mm (0.200 inch), and a relatively short length, typically being in the range from 10 cm to 20 cm. The low system flow resistance is desirable since it permits the flow to be maximized during portions of a procedure when the risk of emboli is at its greatest. The low system flow resistance also allows the use of a variable flow resistance for controlling flow in the system, as described in more detail below. The dimensions of the venous return sheath 910 can be generally the same as those described for the arterial access sheath 605 above. In the venous return sheath, an extension for the hemostasis valve 1010 is not required.

Retrograde Shunt

The shunt 120 can be formed of a single tube or multiple, connected tubes that provide fluid communication between the arterial access catheter 110 and the venous return catheter 115 to provide a pathway for retrograde blood flow therebetween. As shown in FIG. 26, the shunt 120 connects at one end (via connector 127a) to the flow line 615 of the arterial access device 110, and at an opposite end (via connector 127b) to the flow line 915 of the venous return catheter 115.

In an embodiment, the shunt 120 can be formed of at least one tube that communicates with the flow control assembly 125. The shunt 120 can be any structure that provides a fluid pathway for blood flow. The shunt 120 can have a single lumen or it can have multiple lumens. The shunt 120 can be removably attached to the flow control assembly 125, arterial access device 110, and/or venous return device 115. Prior to use, the user can select a shunt 120 with a length that is most appropriate for use with the arterial access location and venous return location. In an embodiment, the shunt 120 can include one or more extension tubes that can be used to vary the length of the shunt 120. The extension tubes can be modularly attached to the shunt 120 to achieve a desired length. The modular aspect of the shunt 120 permits the user to lengthen the shunt 120 as needed depending on the site of venous return. For example, in some patients, the internal jugular vein IJV is small and/or tortuous. The risk of complications at this site may be higher than at some other locations, due to proximity to other anatomic structures. In addition, hematoma in the neck may lead to airway obstruction and/or cerebral vascular complications. Consequently, for such patients it may be desirable to locate the venous return site at a location other than the internal jugular vein IJV, such as the femoral vein. A femoral vein return site may be accomplished percutaneously, with lower risk of serious complication, and also offers an alternative venous access to the central vein if the internal jugular vein IJV is not available. Furthermore, the femoral venous return changes the layout of the reverse flow shunt such that the shunt controls may be located closer to the “working area” of the intervention, where the devices are being introduced and the contrast injection port is located.

In an embodiment, the shunt 120 has an internal diameter of 4.76 mm ( 3/16 inch) and has a length of 40-70 cm. As mentioned, the length of the shunt can be adjusted.

Flow Control Assembly—Regulation and Monitoring of Retrograde Flow

The flow control assembly 125 interacts with the retrograde shunt 120 to regulate and/or monitor the retrograde flow rate from the common carotid artery to the venous return site, such as the internal jugular vein, or to the external receptacle 130. In this regard, the flow control assembly 125 enables the user to achieve higher maximum flow rates than existing systems and to also selectively adjust, set, or otherwise modulate the retrograde flow rate. Various mechanisms can be used to regulate the retrograde flow rate, as described more fully below. The flow control assembly 125 enables the user to configure retrograde blood flow in a manner that is suited for various treatment regimens, as described below.

In general, the ability to control the continuous retrograde flow rate allows the physician to adjust the protocol for individual patients and stages of the procedure. The retrograde blood flow rate will typically be controlled over a range from a low rate to a high rate. The high rate can be at least two fold higher than the low rate, typically being at least three fold higher than the low rate, and often being at least five fold higher than the low rate, or even higher. In an embodiment, the high rate is at least three fold higher than the low rate and in another embodiment the high rate is at least six fold higher than the low rate. While it is generally desirable to have a high retrograde blood flow rate to maximize the extraction of emboli from the carotid arteries, the ability of patients to tolerate retrograde blood flow will vary. Thus, by having a system and protocol which allows the retrograde blood flow rate to be easily modulated, the treating physician can determine when the flow rate exceeds the tolerable level for that patient and set the reverse flow rate accordingly. For patients who cannot tolerate continuous high reverse flow rates, the physician can chose to turn on high flow only for brief, critical portions of the procedure when the risk of embolic debris is highest. At short intervals, for example between 15 seconds and 1 minute, patient tolerance limitations are usually not a factor.

In specific embodiments, the continuous retrograde blood flow rate can be controlled at a base line flow rate in the range from 10 ml/min to 200 ml/min, typically from 20 ml/min to 100 ml/min. These flow rates will be tolerable to the majority of patients. Although flow rate is maintained at the base line flow rate during most of the procedure, at times when the risk of emboli release is increased, the flow rate can be increased above the base line for a short duration in order to improve the ability to capture such emboli. For example, the retrograde blood flow rate can be increased above the base line when the stent catheter is being introduced, when the stent is being deployed, pre- and post-dilatation of the stent, removal of the common carotid artery occlusion, and the like.

The flow rate control system can be cycled between a relatively low flow rate and a relatively high flow rate in order to “flush” the carotid arteries in the region of the carotid bifurcation prior to reestablishing antegrade flow. Such cycling can be established with a high flow rate which can be approximately two to six fold greater than the low flow rate, typically being about three fold greater. The cycles can typically have a length in the range from 0.5 seconds to 10 seconds, usually from 2 seconds to 5 seconds, with the total duration of the cycling being in the range from 5 seconds to 60 seconds, usually from 10 seconds to 30 seconds.

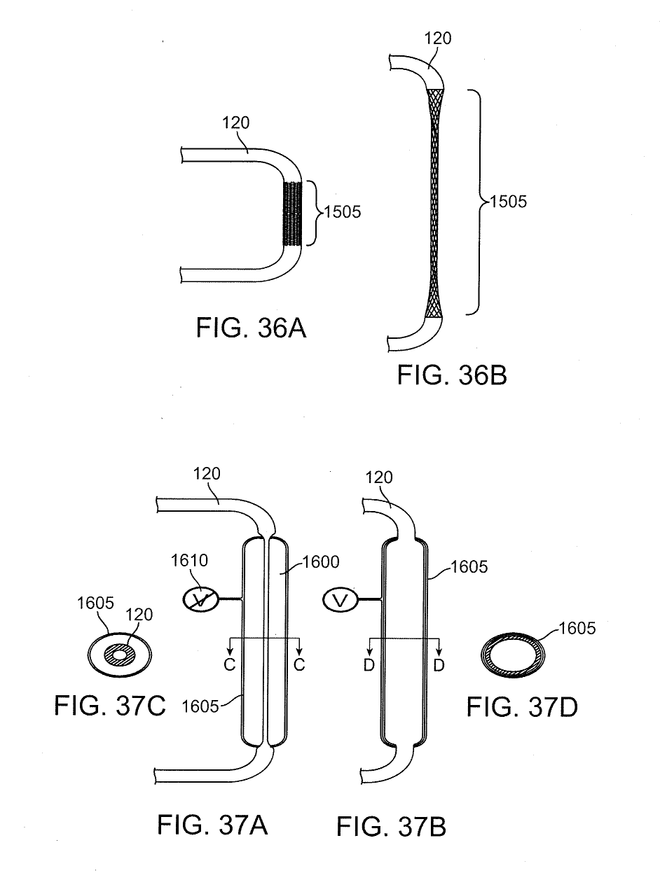

FIG. 33 shows an example of the system 100 with a schematic representation of the flow control assembly 125, which is positioned along the shunt 120 such that retrograde blood flow passes through or otherwise communicates with at least a portion of the flow control assembly 125. The flow control assembly 125 can include various controllable mechanisms for regulating and/or monitoring retrograde flow. The mechanisms can include various means of controlling the retrograde flow, including one or more pumps 1110, valves 1115, syringes 1120 and/or a variable resistance component 1125. The flow control assembly 125 can be manually controlled by a user and/or automatically controlled via a controller 1130 to vary the flow through the shunt 120. For example, varying the flow resistance, the rate of retrograde blood flow through the shunt 120 can be controlled. The controller 1130, which is described in more detail below, can be integrated into the flow control assembly 125 or it can be a separate component that communicates with the components of the flow control assembly 125.

In addition, the flow control assembly 125 can include one or more flow sensors 1135 and/or anatomical data sensors 1140 (described in detail below) for sensing one or more aspects of the retrograde flow. A filter 1145 can be positioned along the shunt 120 for removing emboli before the blood is returned to the venous return site. When the filter 1145 is positioned upstream of the controller) 130, the filter 1145 can prevent emboli from entering the controller 1145 and potentially clogging the variable flow resistance component 1125. It should be appreciated that the various components of the flow control assembly 125 (including the pump 1110, valves 1115, syringes 1120, variable resistance component 1125, sensors 1135/1140, and filter 1145) can be positioned at various locations along the shunt 120 and at various upstream or downstream locations relative to one another. The components of the flow control assembly 125 are not limited to the locations shown in FIG. 33. Moreover, the flow control assembly 125 does not necessarily include all of the components but can rather include various sub-combinations of the components. For example, a syringe could optionally be used within the flow control assembly 125 for purposes of regulating flow or it could be used outside of the assembly for purposes other than flow regulation, such as to introduce fluid such as radiopaque contrast into the artery in an antegrade direction via the shunt 120.

Both the variable resistance component 1125 and the pump 1110 can be coupled to the shunt 120 to control the retrograde flow rate. The variable resistance component 1125 controls the flow resistance, while the pump 1110 provides for positive displacement of the blood through the shunt 120. Thus, the pump can be activated to drive the retrograde flow rather than relying on the perfusion stump pressures of the ECA and ICA and the venous back pressure to drive the retrograde flow. The pump 1110 can be a peristaltic tube pump or any type of pump including a positive displacement pump. The pump 1110 can be activated and deactivated (either manually or automatically via the controller 1130) to selectively achieve blood displacement through the shunt 120 and to control the flow rate through the shunt 120. Displacement of the blood through the shunt 120 can also be achieved in other manners including using the aspiration syringe 1120, or a suction source such as a vacutainer, vaculock syringe, or wall suction may be used. The pump 1110 can communicate with the controller 1130.

One or more flow control valves 1115 can be positioned along the pathway of the shunt. The valve(s) can be manually actuated or automatically actuated (via the controller 1130). The flow control valves 1115 can be, for example one-way valves to prevent flow in the antegrade direction in the shunt 120, check valves, or high pressure valves which would close off the shunt 120, for example during high-pressure contrast injections (which are intended to enter the arterial vasculature in an antegrade direction).

The controller 1130 communicates with components of the system 100 including the flow control assembly 125 to enable manual and/or automatic regulation and/or monitoring of the retrograde flow through the components of the system 100 (including, for example, the shunt 120, the arterial access device 110, the venous return device 115 and the flow control assembly 125). For example, a user can actuate one or more actuators on the controller 1130 to manually control the components of the flow control assembly 125. Manual controls can include switches or dials or similar components located directly on the controller 1130 or components located remote from the controller 1130 such as a foot pedal or similar device. The controller 1130 can also automatically control the components of the system 100 without requiring input from the user. In an embodiment, the user can program software in the controller 1130 to enable such automatic control. The controller 1130 can control actuation of the mechanical portions of the flow control assembly 125. The controller 1130 can include circuitry or programming that interprets signals generated by sensors 1135/1140 such that the controller 1130 can control actuation of the flow control assembly 125 in response to such signals generated by the sensors.

The representation of the controller 1130 in FIG. 33 is merely exemplary. It should be appreciated that the controller 1130 can vary in appearance and structure. The controller 1130 is shown in FIG. 33 as being integrated in a single housing. This permits the user to control the flow control assembly 125 from a single location. It should be appreciated that any of the components of the controller 1130 can be separated into separate housings. Further, FIG. 33 shows the controller 1130 and flow control assembly 125 as separate housings. It should be appreciated that the controller 1130 and flow control regulator 125 can be integrated into a single housing or can be divided into multiple housings or components.

Flow State Indicator(s)

The controller 1130 can include one or more indicators that provides a visual and/or audio signal to the user regarding the state of the retrograde flow. An audio indication advantageously reminds the user of a flow state without requiring the user to visually check the flow controller 1130. The indicator(s) can include a speaker 1150 and/or a light 1155 or any other means for communicating the state of retrograde flow to the user. The controller 1130 can communicate with one or more sensors of the system to control activation of the indicator. Or, activation of the indicator can be tied directly to the user actuating one of the flow control actuators 1165. The indicator need not be a speaker or a light. The indicator could simply be a button or switch that visually indicates the state of the retrograde flow. For example, the button being in a certain state (such as a pressed or down state) may be a visual indication that the retrograde flow is in a high state. Or, a switch or dial pointing toward a particular labeled flow state may be a visual indication that the retrograde flow is in the labeled state.

The indicator can provide a signal indicative of one or more states of the retrograde flow. In an embodiment, the indicator identifies only two discrete states: a state of “high” flow rate and a state of “low” flow rate. In another embodiment, the indicator identifies more than two flow rates, including a “high” flow rate, a “medium” flow rate, and a “low” rate. The indicator can be configured to identify any quantity of discrete states of the retrograde flow or it can identify a graduated signal that corresponds to the state of the retrograde flow. In this regard, the indicator can be a digital or analog meter 1160 that indicates a value of the retrograde flow rate, such as in ml/min or any other units.

In an embodiment, the indicator is configured to indicate to the user whether the retrograde flow rate is in a state of “high” flow rate or a “low” flow rate. For example, the indicator may illuminate in a first manner (e.g., level of brightness) and/or emit a first audio signal when the flow rate is high and then change to a second manner of illumination and/or emit a second audio signal when the flow rate is low. Or, the indicator may illuminate and/or emit an audio signal only when the flow rate is high, or only when the flow rate is low. Given that some patients may be intolerant of a high flow rate or intolerant of a high flow rate beyond an extended period of time, it can be desirable that the indicator provide notification to the user when the flow rate is in the high state. This would serve as a fail safe feature.

In another embodiment, the indicator provides a signal (audio and/or visual) when the flow rate changes state, such as when the flow rate changes from high to low and/or vice-versa. In another embodiment, the indicator provides a signal when no retrograde flow is present, such as when the shunt 120 is blocked or one of the stopcocks in the shunt 120 is closed.

Flow Rate Actuators

The controller 1130 can include one or more actuators that the user can press, switch, manipulate, or otherwise actuate to regulate the retrograde flow rate and/or to monitor the flow rate. For example, the controller 1130 can include a flow control actuator 1165 (such as one or more buttons, knobs, dials, switches, etc.) that the user can actuate to cause the controller to selectively vary an aspect of the reverse flow. For example, in the illustrated embodiment, the flow control actuator 1165 is a knob that can be turned to various discrete positions each of which corresponds to the controller 1130 causing the system 100 to achieve a particular retrograde flow state. The states include, for example, (a) OFF; (b) LO-FLOW; (c) HI-FLOW; and (d) ASPIRATE. It should be appreciated that the foregoing states are merely exemplary and that different states or combinations of states can be used. The controller 1130 achieves the various retrograde flow states by interacting with one or more components of the system, including the sensor(s), valve(s), variable resistance component, and/or pump(s). It should be appreciated that the controller 1130 can also include circuitry and software that regulates the retrograde flow rate and/or monitors the flow rate such that the user wouldn't need to actively actuate the controller 1130.

The OFF state corresponds to a state where there is no retrograde blood flow through the shunt 120. When the user sets the flow control actuator 1165 to OFF, the controller 1130 causes the retrograde flow to cease, such as by shutting off valves or closing a stop cock in the shunt 120. The LO-FLOW and HI-FLOW states correspond to a low retrograde flow rate and a high retrograde flow rate, respectively. When the user sets the flow control actuator 1165 to LO-FLOW or HI-FLOW, the controller 1130 interacts with components of the flow control regulator 125 including pump(s) 1110, valve(s) 1115 and/or variable resistance component 1125 to increase or decrease the flow rate accordingly. Finally, the ASPIRATE state corresponds to opening the circuit to a suction source, for example a vacutainer or suction unit, if active retrograde flow is desired.

The system can be used to vary the blood flow between various states including an active state, a passive state, an aspiration state, and an off state. The active state corresponds to the system using a means that actively drives retrograde blood flow. Such active means can include, for example, a pump, syringe, vacuum source, etc. The passive state corresponds to when retrograde blood flow is driven by the perfusion stump pressures of the ECA and ICA and possibly the venous pressure. The aspiration state corresponds to the system using a suction source, for example a vacutainer or suction unit, to drive retrograde blood flow. The off state corresponds to the system having zero retrograde blood flow such as the result of closing a stopcock or valve. The low and high flow rates can be either passive or active flow states. In an embodiment, the particular value (such as in ml/min) of either the low flow rate and/or the high flow rate can be predetermined and/or pre-programmed into the controller such that the user does not actually set or input the value. Rather, the user simply selects “high flow” and/or “low flow” (such as by pressing an actuator such as a button on the controller 1130) and the controller 1130 interacts with one or more of the components of the flow control assembly 125 to cause the flow rate to achieve the predetermined high or low flow rate value. In another embodiment, the user sets or inputs a value for low flow rate and/or high flow rate such as into the controller. In another embodiment, the low flow rate and/or high flow rate is not actually set. Rather, external data (such as data from the anatomical data sensor 1140) is used as the basis for affects the flow rate.

The flow control actuator 1165 can be multiple actuators, for example one actuator, such as a button or switch, to switch state from LO-FLOW to HI-FLOW and another to close the flow loop to OFF, for example during a contrast injection where the contrast is directed antegrade into the carotid artery. In an embodiment, the flow control actuator 1165 can include multiple actuators. For example, one actuator can be operated to switch flow rate from low to high, another actuator can be operated to temporarily stop flow, and a third actuator (such as a stopcock) can be operated for aspiration using a syringe. In another example, one actuator is operated to switch to LO-FLOW and another actuator is operated to switch to HI-FLOW. Or, the flow control actuator 1165 can include multiple actuators to switch states from LO-FLOW to HI-FLOW and additional actuators for fine-tuning flow rate within the high flow state and low flow state. Upon switching between LO-FLOW and HI-FLOW, these additional actuators can be used to fine-tune the flow rates within those states. Thus, it should be appreciated that within each state (i.e. high flow state and low flow states) a variety of flow rates can be dialed in and fine-tuned. A wide variety of actuators can be used to achieve control over the state of flow.

The controller 1130 or individual components of the controller 1130 can be located at various positions relative to the patient and/or relative to the other components of the system 100. For example, the flow control actuator 1165 can be located near the hemostasis valve where any interventional tools are introduced into the patient in order to facilitate access to the flow control actuator 1165 during introduction of the tools. The location may vary, for example, based on whether a transfemoral or a transcervical approach is used. The controller 1130 can have a wireless connection to the remainder of the system 100 and/or a wired connection of adjustable length to permit remote control of the system 100. The controller 1130 can have a wireless connection with the flow control regulator 125 and/or a wired connection of adjustable length to permit remote control of the flow control regulator 125. The controller 1130 can also be integrated in the flow control regulator 125. Where the controller 1130 is mechanically connected to the components of the flow control assembly 125, a tether with mechanical actuation capabilities can connect the controller 1130 to one or more of the components. In an embodiment, the controller 1130 can be positioned a sufficient distance from the system 100 to permit positioning the controller 1130 outside of a radiation field when fluoroscopy is in use.

The controller 1130 and any of its components can interact with other components of the system (such as the pump(s), sensor(s), shunt, etc) in various manners. For example, any of a variety of mechanical connections can be used to enable communication between the controller 1130 and the system components. Alternately, the controller 1130 can communicate electronically or magnetically with the system components. Electro-mechanical connections can also be used. The controller 1130 can be equipped with control software that enables the controller to implement control functions with the system components. The controller itself can be a mechanical, electrical or electro-mechanical device. The controller can be mechanically, pneumatically, or hydraulically actuated or electromechanically actuated (for example in the case of solenoid actuation of flow control state). The controller 1130 can include a computer, computer processor, and memory, as well as data storage capabilities.

Sensor(s)

As mentioned, the flow control assembly 125 can include or interact with one or more sensors, which communicate with the system 100 and/or communicate with the patient's anatomy. Each of the sensors can be adapted to respond to a physical stimulus (including, for example, heat, light, sound, pressure, magnetism, motion, etc.) and to transmit a resulting signal for measurement or display or for operating the controller 1130. In an embodiment, the flow sensor 1135 interacts with the shunt 120 to sense an aspect of the flow through the shunt 120, such as flow velocity or volumetric rate of blood flow. The flow sensor 1135 could be directly coupled to a display that directly displays the value of the volumetric flow rate or the flow velocity. Or the flow sensor 1135 could feed data to the controller 1130 for display of the volumetric flow rate or the flow velocity.

The type of flow sensor 1135 can vary. The flow sensor 1135 can be a mechanical device, such as a paddle wheel, flapper valve, rolling ball, or any mechanical component that responds to the flow through the shunt 120. Movement of the mechanical device in response to flow through the shunt 120 can serve as a visual indication of fluid flow and can also be calibrated to a scale as a visual indication of fluid flow rate. The mechanical device can be coupled to an electrical component. For example, a paddle wheel can be positioned in the shunt 120 such that fluid flow causes the paddle wheel to rotate, with greater rate of fluid flow causing a greater speed of rotation of the paddle wheel. The paddle wheel can be coupled magnetically to a Hall-effect sensor to detect the speed of rotation, which is indicative of the fluid flow rate through the shunt 120.

In an embodiment, the flow sensor 1135 is an ultrasonic or electromagnetic flow meter, which allows for blood flow measurement without contacting the blood through the wall of the shunt 120. An ultrasonic or electromagnetic flow meter can be configured such that it does not have to contact the internal lumen of the shunt 120. In an embodiment, the flow sensor 1135 at least partially includes a Doppler flow meter, such as a Transonic flow meter, that measures fluid flow through the shunt 120. It should be appreciated that any of a wide variety of sensor types can be used including an ultrasound flow meter and transducer. Moreover, the system can include multiple sensors.

The system 100 is not limited to using a flow sensor 1135 that is positioned in the shunt 120 or a sensor that interacts with the venous return device 115 or the arterial access device 110. For example, an anatomical data sensor 1140 can communicate with or otherwise interact with the patient's anatomy such as the patient's neurological anatomy. In this manner, the anatomical data sensor 1140 can sense a measurable anatomical aspect that is directly or indirectly related to the rate of retrograde flow from the carotid artery. For example, the anatomical data sensor 1140 can measure blood flow conditions in the brain, for example the flow velocity in the middle cerebral artery, and communicate such conditions to a display and/or to the controller 1130 for adjustment of the retrograde flow rate based on predetermined criteria. In an embodiment, the anatomical data sensor 1140 comprises a transcranial Doppler ultrasonography (TCD), which is an ultrasound test that uses reflected sound waves to evaluate blood as it flows through the brain. Use of TCD results in a TCD signal that can be communicated to the controller 1130 for controlling the retrograde flow rate to achieve or maintain a desired TCD profile. The anatomical data sensor 1140 can be based on any physiological measurement, including reverse flow rate, blood flow through the middle cerebral artery, TCD signals of embolic particles, or other neuromonitoring signals.

In an embodiment, the system 100 comprises a closed-loop control system. In the closed-loop control system, one or more of the sensors (such as the flow sensor 1135 or the anatomical data sensor 1140) senses or monitors a predetermined aspect of the system 100 or the anatomy (such as, for example, reverse flow rate and/or neuromonitoring signal). The sensor(s) feed relevant data to the controller 1130, which continuously adjusts an aspect of the system as necessary to maintain a desired retrograde flow rate. The sensors communicate feedback on how the system 100 is operating to the controller 1130 so that the controller 1130 can translate that data and actuate the components of the flow control regulator 125 to dynamically compensate for disturbances to the retrograde flow rate. For example, the controller 1130 may include software that causes the controller 1130 to signal the components of the flow control assembly 125 to adjust the flow rate such that the flow rate is maintained at a constant state despite differing blood pressures from the patient. In this embodiment, the system 100 need not rely on the user to determine when, how long, and/or what value to set the reverse flow rate in either a high or low state. Rather, software in the controller 1130 can govern such factors. In the closed loop system, the controller 1130 can control the components of the flow control assembly 125 to establish the level or state of retrograde flow (either analog level or discreet state such as high, low, baseline, medium, etc.) based on the retrograde flow rate sensed by the sensor 1135.

In an embodiment, the anatomical data sensor 1140 (which measures a physiologic measurement in the patient) communicates a signal to the controller 1130, which adjusts the flow rate based on the signal. For example the physiological measurement may be based on flow velocity through the MCA, TCD signal, or some other cerebral vascular signal. In the case of the TCD signal, TCD may be used to monitor cerebral flow changes and to detect microemboli. The controller 1130 may adjust the flow rate to maintain the TCD signal within a desired profile. For example, the TCD signal may indicate the presence of microemboli (“TCD hits”) and the controller 1130 can adjust the retrograde flow rate to maintain the TCD hits below a threshold value of hits. (See, Ribo, et al., “Transcranial Doppler Monitoring of Transcervical Carotid Stenting with Flow Reversal Protection: A Novel Carotid Revascularization Technique”, Stroke 2006, 37, 2846-2849; Shekel, et al., “Experience of 500 Cases of Neurophysiological Monitoring in Carotid Endarterectomy”, Acta Neurochir, 2007, 149:681-689, which are incorporated by reference in their entirety.

In the case of the MCA flow, the controller 1130 can set the retrograde flow rate at the “maximum” flow rate that is tolerated by the patient, as assessed by perfusion to the brain. The controller 1130 can thus control the reverse flow rate to optimize the level of protection for the patient without relying on the user to intercede. In another embodiment, the feedback is based on a state of the devices in the system 100 or the interventional tools being used. For example, a sensor may notify the controller 1130 when the system 100 is in a high risk state, such as when an interventional catheter is positioned in the sheath 605. The controller 1130 then adjusts the flow rate to compensate for such a state.

The controller 1130 can be used to selectively augment the retrograde flow in a variety of manners. For example, it has been observed that greater reverse flow rates may cause a resultant greater drop in blood flow to the brain, most importantly the ipsilateral MCA, which may not be compensated enough with collateral flow from the Circle of Willis. Thus a higher reverse flow rate for an extended period of time may lead to conditions where the patient's brain is not getting enough blood flow, leading to patient intolerance as exhibited by neurologic symptoms. Studies show that MCA blood velocity less than 10 cm/sec is a threshold value below which patient is at risk for neurological blood deficit. There are other markers for monitoring adequate perfusion to the brains, such as EEG signals. However, a high flow rate may be tolerated even up to a complete stoppage of MCA flow for a short period, up to about 15 seconds to 1 minute.

Thus, the controller 1130 can optimize embolic debris capture by automatically increasing the reverse flow only during limited time periods which correspond to periods of heightened risk of emboli generation during a procedure. These periods of heightened risk include the period of time while an interventional device (such as a dilatation balloon for pre or post stenting dilatation or a stent delivery device) crosses the plaque P. Another period is during an interventional maneuver such as deployment of the stent or inflation and deflation of the balloon pre- or post-dilatation. A third period is during injection of contrast for angiographic imaging of treatment area. During lower risk periods, the controller can cause the reverse flow rate to revert to a lower, baseline level. This lower level may correspond to a low reverse flow rate in the ICA, or even slight antegrade flow in those patients with a high ECA to ICA perfusion pressure ratio.

In a flow regulation system where the user manually sets the state of flow, there is risk that the user may not pay attention to the state of retrograde flow (high or low) and accidentally keep the circuit on high flow. This may then lead to adverse patient reactions. In an embodiment, as a safety mechanism, the default flow rate is the low flow rate. This serves as a fail safe measure for patient's that are intolerant of a high flow rate. In this regard, the controller 1130 can be biased toward the default rate such that the controller causes the system to revert to the low flow rate after passage of a predetermined period of time of high flow rate. The bias toward low flow rate can be achieved via electronics or software, or it can be achieved using mechanical components, or a combination thereof. In an embodiment, the flow control actuator 1165 of the controller 1130 and/or valve(s) 1115 and/or pump(s) 1110 of the flow control regulator 125 are spring loaded toward a state that achieves a low flow rate. The controller 1130 is configured such that the user may over-ride the controller 1130 such as to manually cause the system to revert to a state of low flow rate if desired.

In another safety mechanism, the controller 1130 includes a timer 1170 (FIG. 33) that keeps time with respect to how long the flow rate has been at a high flow rate. The controller 1130 can be programmed to automatically cause the system 100 to revert to a low flow rate after a predetermined time period of high flow rate, for example after 15, 30, or 60 seconds or more of high flow rate. After the controller reverts to the low flow rate, the user can initiate another predetermined period of high flow rate as desired. Moreover, the user can override the controller 1130 to cause the system 100 to move to the low flow rate (or high flow rate) as desired.

In an exemplary procedure, embolic debris capture is optimized while not causing patient tolerance issues by initially setting the level of retrograde flow at a low rate, and then switching to a high rate for discreet periods of time during critical stages in the procedure. Alternately, the flow rate is initially set at a high rate, and then verifying patient tolerance to that level before proceeding with the rest of the procedure. If the patient shows signs of intolerance, the retrograde flow rate is lowered. Patient tolerance may be determined automatically by the controller based on feedback from the anatomical data sensor 1140 or it may be determined by a user based on patient observation. The adjustments to the retrograde flow rate may be performed automatically by the controller or manually by the user. Alternately, the user may monitor the flow velocity through the middle cerebral artery (MCA), for example using TCD, and then to set the maximum level of reverse flow which keeps the MCA flow velocity above the threshold level. In this situation, the entire procedure may be done without modifying the state of flow. Adjustments may be made as needed if the MCA flow velocity changes during the course of the procedure, or the patient exhibits neurologic symptoms.

Exemplary Mechanisms to Regulate Flow

The system 100 is adapted to regulate retrograde flow in a variety of manners. Any combination of the pump 1110, valve 1115, syringe 1120, and/or variable resistance component 1125 can be manually controlled by the user or automatically controlled via the controller 1130 to adjust the retrograde flow rate. Thus, the system 100 can regulate retrograde flow in various manners, including controlling an active flow component (e.g., pump, syringe, etc.), reducing the flow restriction, switching to an aspiration source (such as a pre-set VacLock syringe, Vacutainer, suction system, or the like), or any combination thereof.

In the situation where an external receptacle or reservoir is used, the retrograde flow may be augmented in various manners. The reservoir has a head height comprised of the height of the blood inside the reservoir and the height of the reservoir with respect to the patient. Reverse flow into the reservoir may be modulated by setting the reservoir height to increase or decrease the amount of pressure gradient from the CCA to the reservoir. In an embodiment, the reservoir is raised to increase the reservoir pressure to a pressure that is greater than venous pressure. Or, the reservoir can be positioned below the patient, such as down to a level of the floor, to lower the reservoir pressure to a pressure below venous or atmospheric pressure.

The variable flow resistance in shunt 120 may be provided in a wide variety of ways. In this regard, flow resistance component 1125 can cause a change in the size or shape of the shunt to vary flow conditions and thereby vary the flow rate. Or, the flow resistance component 1125 can re-route the blood flow through one or more alternate flow pathways in the shunt to vary the flow conditions. Some exemplary embodiments of the flow resistance component 1125 are now described.

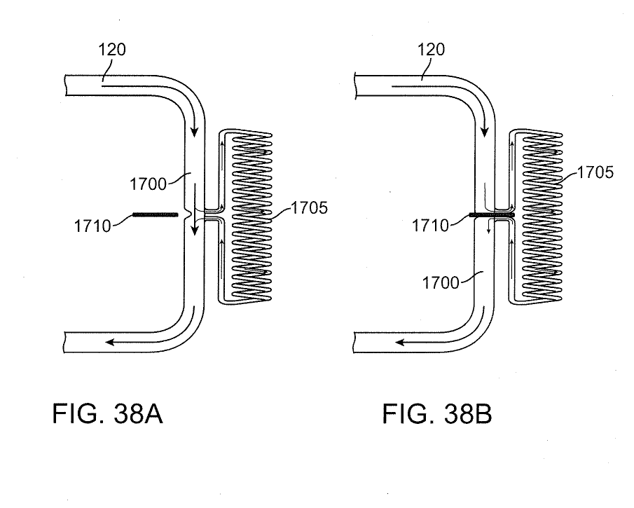

As shown in FIGS. 34A, 34B, 34C, and 34D, in an embodiment the shunt 120 has an inflatable bladder 1205 formed along a portion of its interior lumen. As shown in FIGS. 34A and 34C, when the bladder 1205 is deflated, the inner lumen of the shunt 120 remains substantially unrestricted, providing for a low resistance flow. By inflating the bladder 1205, however, as shown in FIGS. 34B and 34D, the flow lumen can be greatly restricted, thus greatly increasing the flow resistance and reducing the flow rate of atrial blood to the venous vasculature. The controller 1130 can control inflation/deflation of the bladder 1205 or it can be controlled manually by the user.

Rather than using an inflatable internal bladder, as shown in FIGS. 34A-34D, the cross-sectional area of the lumen in the shunt 120 may be decreased by applying an external force, such as flattening the shunt 120 with a pair of opposed plates 1405, as shown in FIGS. 35A-35D. The opposed plates are adapted to move toward and away from one another with the shunt 120 positioned between the plates. When the plates 1405 are spaced apart, as shown in FIGS. 35A and 35C, the lumen of the shunt 120 remains unrestricted. When the plates 1405 are closed on the shunt 120, as shown in FIGS. 35B and 35D, in contrast, the plates 1405 constrict the shunt 120. In this manner, the lumen remaining in shunt 120 can be greatly decreased to increase flow resistance through the shunt. The controller 1130 can control movement of the plates 1405 or such movement can be controlled manually by the user.

Referring now to FIGS. 36A and 36B, the available cross-sectional area of the shunt 120 can also be restricted by axially elongating a portion 1505 of the shunt 120. Prior to axial elongation, the portion 1505 will be generally unchanged, providing a full luminal flow area in the portion 1505, as shown in FIG. 36A. By elongating the portion 1505, however, as shown in FIG. 36B, the internal luminal area of the shunt 120 in the portion 1505 can be significantly decreased and the length increased, both of which have the effect of increasing the flow resistance. When employing axial elongation to reduce the luminal area of shunt 120, it will be advantageous to employ a mesh or braid structure in the shunt at least in the portion 1505. The mesh or braid structure provides the shunt 120 with a pliable feature that facilitates axial elongation without breaking. The controller 1130 can control elongation of the shunt 120 or such it can be controlled manually by the user.

Referring now to FIGS. 37A-37D, instead of applying an external force to reduce the cross-sectional area of shunt 120, a portion of the shunt 120 can be made with a small diameter to begin with, as shown in FIGS. 37A and 37C. The shunt 120 passes through a chamber 1600 which is sealed at both ends. A vacuum is applied within the chamber 1600 exterior of the shunt 120 to cause a pressure gradient. The pressure gradient cause the shunt 120 to increase in size within the chamber 120, as shown in FIGS. 37B and 37D. The vacuum may be applied in a receptacle 1605 attached to a vacuum source 1610. Conversely, a similar system may be employed with a shunt 120 whose resting configuration is in the increased size. Pressure may be applied to the chamber to shrink or flatten the shunt to decrease the flow resistance. The controller 1130 can control the vacuum or it can be controlled manually by the user.

As yet another alternative, the flow resistance through shunt 120 may be changed by providing two or more alternative flow paths. As shown in FIG. 38A, the flow through shunt 120 passes through a main lumen 1700 as well as secondary lumen 1705. The secondary lumen 1705 is longer and/or has a smaller diameter than the main lumen 1700. Thus, the secondary lumen 1705 has higher flow resistance than the main lumen 1700. By passing the blood through both these lumens, the flow resistance will be at a minimum. Blood is able to flow through both lumens 1700 and 1705 due to the pressure drop created in the main lumen 1700 across the inlet and outlet of the secondary lumen 1705. This has the benefit of preventing stagnant blood. As shown in FIG. 38B, by blocking flow through the main lumen 1700 of shunt 120, the flow can be diverted entirely to the secondary lumen 1705, thus increasing the flow resistance and reducing the blood flow rate. It will be appreciated that additional flow lumens could also be provided in parallel to allow for a three, four, or more discrete flow resistances. The shunt 120 may be equipped with a valve 1710 that controls flow to the main lumen 1700 and the secondary lumen 1705 with the valve 1710 being controlled by the controller 1130 or being controlled manually by the user. The embodiment of FIGS. 38A and 38B has an advantage in that this embodiment in that it does not require as small of lumen sizes to achieve desired retrograde flow rates as some of the other embodiments of variable flow resistance mechanisms. This is a benefit in blood flow lines in that there is less chance of clogging and causing clots in larger lumen sizes than smaller lumen sizes.

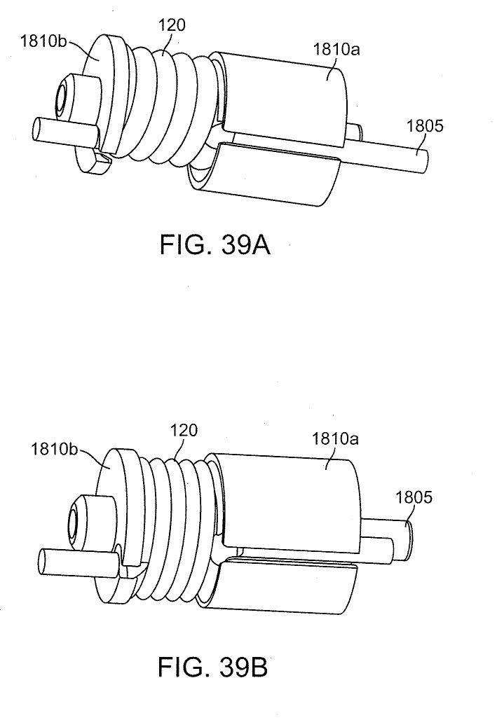

The shunt 120 can also be arranged in a variety of coiled configurations which permit external compression to vary the flow resistance in a variety of ways. Arrangement of a portion of the shunt 120 in a coil contains a long section of the shunt in a relatively small area. This allows compression of a long length of the shunt 120 over a small space. As shown in FIGS. 39A and 39B, a portion of the shunt 120 is wound around a dowel 1805 to form a coiled region. The dowel 1805 has plates 1810a and 1810b which can move toward and away from each other in an axial direction. When plates 1810a and 1810b are moved away from each other, the coiled portion of the shunt 105 is uncompressed and flow resistance is at a minimum. The shunt 120 is large diameter, so when the shunt is non-compressed, the flow resistance is low, allowing a high-flow state. To down-regulate the flow, the two plates 1810a and 1810b are pushed together, compressing the coil of shunt 120. By moving the plates 1810a and 1810b together, as shown in FIG. 39B, the coiled portion of the shunt 120 is compressed to increase the flow resistance. The controller 1130 can control the plates or they can be controlled manually by the user.

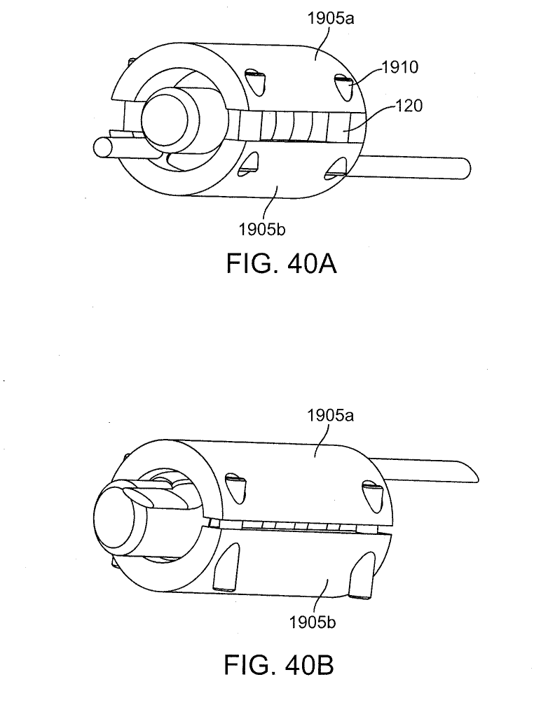

A similar compression apparatus is shown in FIGS. 40A and 40B. In this configuration, the coiled shunt 120 is encased between two movable cylinder halves 1905a and 1905b. The halves 1905a and 1905b can slide along dowel pins 1910 to move toward and away from one another. When the cylinder halves 1905 are moved apart, the coiled shunt 120 is uncompressed and flow resistance is at a minimum. When the cylinder halves 1905 are brought together, the coiled shunt 120 is compressed circumferentially to increase flow resistance. The controller 1130 can control the halves 1905 or they can be controlled manually by the user.

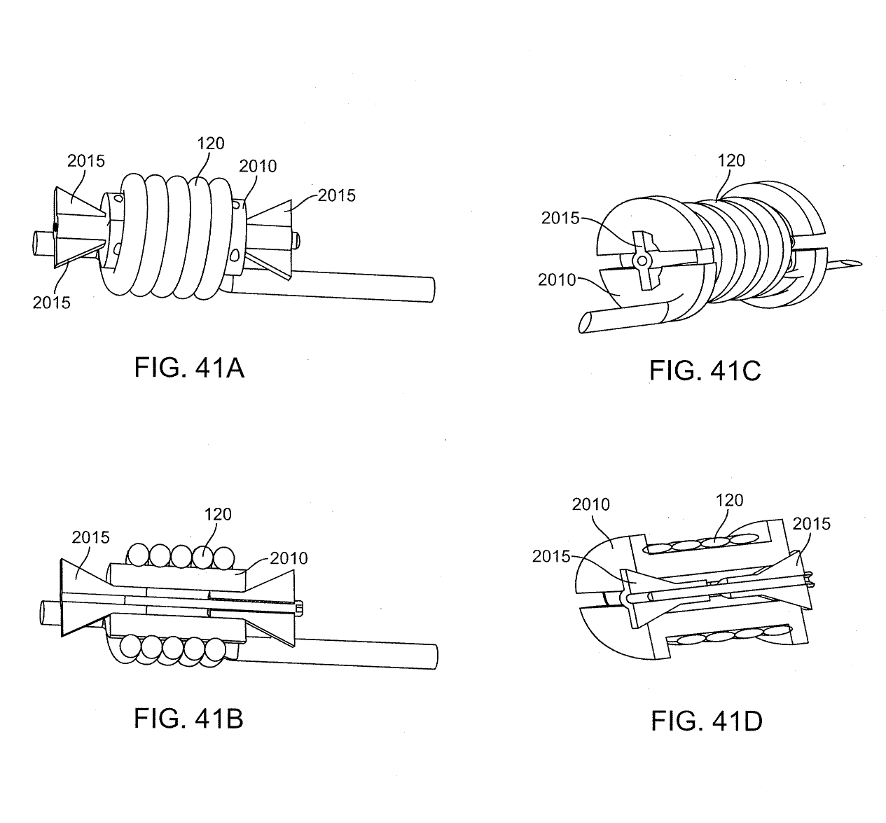

As shown in FIGS. 41A through 41D, the shunt 120 may also be wound around an axially split mandrel 2010 having wedge elements 2015 on opposed ends. By axially translating wedge elements 2015 in and out of the split mandrel 2010, the split portions of the mandrel are opened and closed relative to one another, causing the coil of tubing to be stretched (when the mandrel portions 2010 are spread apart, FIG. 41C, 41D) or relaxed (when the mandrel portions 2010 are closed, FIG. 41A, 41B.) Thus, when the wedge elements 2015 are spaced apart, as shown in FIGS. 41A and 41B, the outward pressure on the shunt 120 is at a minimum and the flow resistance is also at a minimum. By driving the wedge elements 2015 inwardly, as shown in FIGS. 41C and 41D, the split mandrel halves 2020 are forced apart and the coil of shunt 120 is stretched. This has the dual effect of decreasing the cross sectional area of the shunt and lengthening the shunt in the coiled region, both of which lead to increased flow resistance.

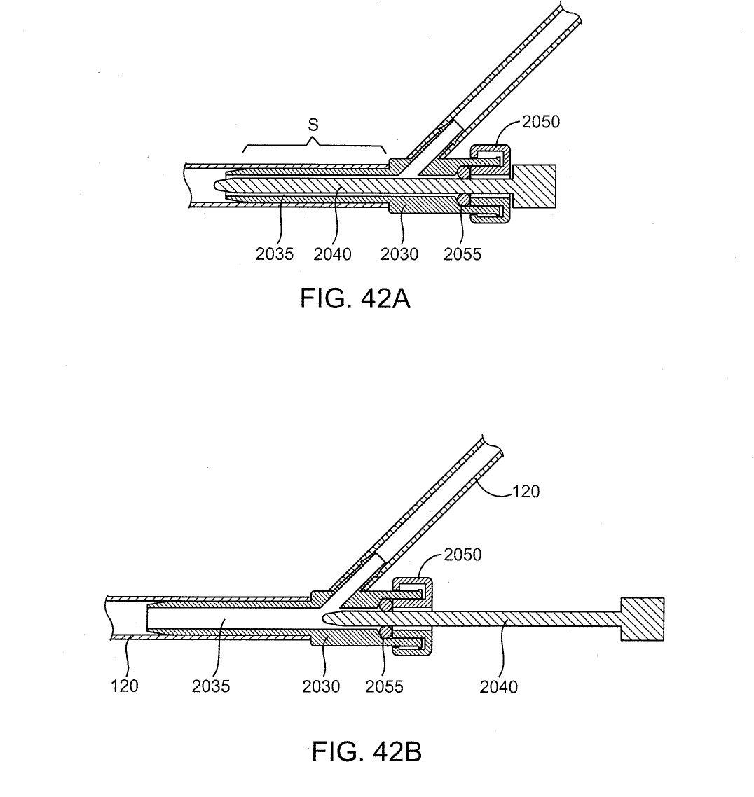

FIGS. 42A and 42B show an embodiment of the variable resistance component 1125 that uses a dowel to vary the resistance to flow. A housing 2030 is inserted into a section of the shunt 120. The housing 2030 has an internal lumen 2035 that is contiguous with the internal lumen of the shunt 120. A dowel 2040 can move into and out of a portion of the internal lumen 2035. As shown in FIG. 42A, when the dowel 2040 is inserted into the internal lumen 2035, the internal lumen 2035 is annular with a cross-sectional area that is much smaller than the cross-sectional area of the internal lumen 2035 when the dowel is not present. Thus, flow resistance increases when the dowel 2040 is positioned in the internal lumen 2035. The annular internal lumen 2035 has a length S that can be varied by varying the portion of the dowel 2040 that is inserted into the lumen 2035. Thus, as more of the dowel 2040 is inserted, the length S of the annular lumen 2035 increases and vice-versa. This can be used to vary the level of flow resistance caused by the presence of the dowel 2040.

The dowel 2040 enters the internal lumen 2035 via a hemostasis valve in the housing 2030. A cap 2050 and an O-ring 2055 provide a sealing engagement that seals the housing 2030 and dowel 2040 against leakage. The cap 2050 may have a locking feature, such as threads, that can be used to lock the cap 2050 against the housing 2030 and to also fix the position of the dowel 2040 in the housing 2040. When the cap 2050 is locked or tightened, the cap 2050 exerts pressure against the O-ring 2055 to tighten it against the dowel 2040 in a sealed engagement. When the cap 2050 is unlocked or untightened, the dowel 2040 is free to move in and out of the housing 2030.

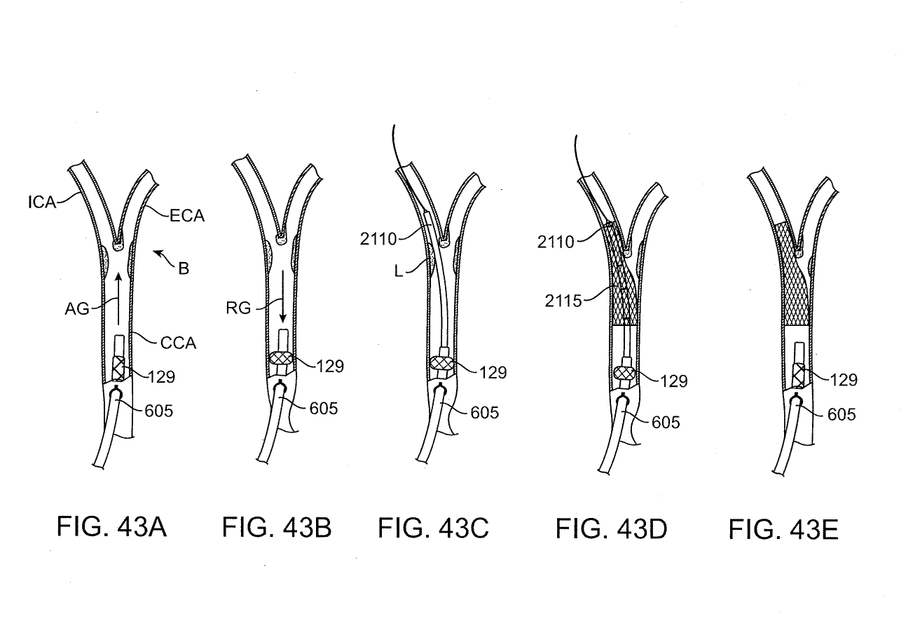

Referring now to FIGS. 43A-43E, flow through the carotid artery bifurcation at different stages of the methods of the present disclosure will be described. Initially, as shown in FIG. 43A, the distal sheath 605 of the arterial access device 110 is introduced into the common carotid artery CCA. As mentioned, entry into the common carotid artery CCA can be via a transcervical or transfemoral approach. After the sheath 605 of the arterial access device 110 has been introduced into the common carotid artery CCA, the blood flow will continue in antegrade direction AG with flow from the common carotid artery entering both the internal carotid artery ICA and the external carotid artery ECA, as shown in FIG. 43A.

The venous return device 115 is then inserted into a venous return site, such as the internal jugular vein IJV (not shown in FIGS. 43A-43E). The shunt 120 is used to connect the flow lines 615 and 915 of the arterial access device 110 and the venous return device 115, respectively. In this manner, the shunt 120 provides a passageway for retrograde flow from the atrial access device 110 to the venous return device 115. In another embodiment, the shunt 120 connects to an external receptacle 130 rather than to the venous return device 115.

Once all components of the system are in place and connected, flow through the common carotid artery CCA is stopped, typically using the occlusion element 129 as shown in FIG. 43B. The occlusion element 129 is expanded at a location proximal to the distal opening of the sheath 605 to occlude the CCA. Alternately, a tourniquet or other external vessel occlusion device can be used to occlude the common carotid artery CCA to stop flow therethrough. In an alternative embodiment, the occlusion element 129 is introduced on second occlusion device 112 separate from the distal sheath 605 of the arterial access device 110. The ECA may also be occluded with a separate occlusion element, either on the same device 110 or on a separate occlusion device.

At that point retrograde flow RG from the external carotid artery ECA and internal carotid artery ICA will begin and will flow through the sheath 605, the flow line 615, the shunt 120, and into the venous return device 115 via the flow line 915. The flow control assembly125 regulates the retrograde flow as described above. FIG. 43B shows the occurrence of retrograde flow RG. While the retrograde flow is maintained, a stent delivery catheter 2110 is introduced into the sheath 605, as shown in FIG. 43C. The stent delivery catheter 2110 is introduced into the sheath 605 through the hemostasis valve 615 and the proximal extension 610 (not shown in FIGS. 43A-43E) of the arterial access device 110. The stent delivery catheter 2110 is advanced into the internal carotid artery ICA and a stent 2115 deployed at the bifurcation B, as shown in FIG. 43D.

The rate of retrograde flow can be increased during periods of higher risk for emboli generation for example while the stent delivery catheter 2110 is being introduced and optionally while the stent 2115 is being deployed. The rate of retrograde flow can be increased also during placement and expansion of balloons for dilatation prior to or after stent deployment. An atherectomy can also be performed before stenting under retrograde flow.

Still further optionally, after the stent 2115 has been expanded, the bifurcation B can be flushed by cycling the retrograde flow between a low flow rate and high flow rate. The region within the carotid arteries where the stent has been deployed or other procedure performed may be flushed with blood prior to reestablishing normal blood flow. In particular, while the common carotid artery remains occluded, a balloon catheter or other occlusion element may be advanced into the internal carotid artery and deployed to fully occlude that artery. The same maneuver may also be used to perform a post-deployment stent dilatation, which is typically done currently in self-expanding stent procedures. Flow from the common carotid artery and into the external carotid artery may then be reestablished by temporarily opening the occluding means present in the artery. The resulting flow will thus be able to flush the common carotid artery which saw slow, turbulent, or stagnant flow during carotid artery occlusion into the external carotid artery. In addition, the same balloon may be positioned distally of the stent during reverse flow and forward flow then established by temporarily relieving occlusion of the common carotid artery and flushing. Thus, the flushing action occurs in the stented area to help remove loose or loosely adhering embolic debris in that region.

Optionally, while flow from the common carotid artery continues and the internal carotid artery remains blocked, measures can be taken to further loosen emboli from the treated region. For example, mechanical elements may be used to clean or remove loose or loosely attached plaque or other potentially embolic debris within the stent, thrombolytic or other fluid delivery catheters may be used to clean the area, or other procedures may be performed. For example, treatment of in-stent restenosis using balloons, atherectomy, or more stents can be performed under retrograde flow In another example, the occlusion balloon catheter may include flow or aspiration lumens or channels which open proximal to the balloon. Saline, thrombolytics, or other fluids may be infused and/or blood and debris aspirated to or from the treated area without the need for an additional device. While the emboli thus released will flow into the external carotid artery, the external carotid artery is generally less sensitive to emboli release than the internal carotid artery. By prophylactically removing potential emboli which remain, when flow to the internal carotid artery is reestablished, the risk of emboli release is even further reduced. The emboli can also be released under retrograde flow so that the emboli flows through the shunt 120 to the venous system, a filter in the shunt 120, or the receptacle 130.

After the bifurcation has been cleared of emboli, the occlusion element 129 or alternately the tourniquet 2105 can be released, reestablishing antegrade flow, as shown in FIG. 43E. The sheath 605 can then be removed.

A closing element, such as a self-closing element, may be deployed about the penetration in the wall of the common carotid artery prior to withdrawing the sheath 605 at the end of the procedure. Usually, the closing element will be deployed at or near the beginning of the procedure, but optionally, the closing element could be deployed as the sheath is being withdrawn, often being released from a distal end of the sheath onto the wall of the common carotid artery. Use of the self-closing element is advantageous since it affects substantially the rapid closure of the penetration in the common carotid artery as the sheath is being withdrawn. Such rapid closure can reduce or eliminate unintended blood loss either at the end of the procedure or during accidental dislodgement of the sheath. In addition, such a self-closing element may reduce the risk of arterial wall dissection during access. Further, the closing element may be configured to exert a frictional or other retention force on the sheath during the procedure. Such a retention force is advantageous and can reduce the chance of accidentally dislodging the sheath during the procedure. A self-closing element eliminates the need for vascular surgical closure of the artery with suture after sheath removal, reducing the need for a large surgical field and greatly reducing the surgical skill required for the procedure.

In another embodiment, carotid artery stenting may be performed after the sheath is placed and an occlusion balloon catheter deployed in the external carotid artery. The stent having a side hole or other element intended to not block the ostium of the external carotid artery may be delivered through the sheath with a guidewire or a shaft of an external carotid artery occlusion balloon received through the side hole. Thus, as the stent is advanced, typically by a catheter being introduced over a guidewire which extends into the internal carotid artery, the presence of the catheter shaft in the side hole will ensure that the side hole becomes aligned with the ostium to the external carotid artery as the stent is being advanced. When an occlusion balloon is deployed in the external carotid artery, the side hole prevents trapping the external carotid artery occlusion balloon shaft with the stent which is a disadvantage of the other flow reversal systems. This approach also avoids “jailing” the external carotid artery, and if the stent is covered with a graft material, avoids blocking flow to the external carotid artery.

In an embodiment, the user first determines whether any periods of heightened risk of emboli generation may exist during the procedure. As mentioned, some exemplary periods of heightened risk include (1) during periods when the plaque P is being crossed by a device; (2) during an interventional procedure, such as during delivery of a stent or during inflation or deflation of a balloon catheter or guidewire; (3) during injection or contrast. The foregoing are merely examples of periods of heightened risk. During such periods, the user sets the retrograde flow at a high rate for a discreet period of time. At the end of the high risk period, or if the patient exhibits any intolerance to the high flow rate, then the user reverts the flow state to baseline flow. If the system has a timer, the flow state automatically reverts to baseline flow after a set period of time. In this case, the user may re-set the flow state to high flow if the procedure is still in a period of heightened embolic risk.

In another embodiment, if the patient exhibits an intolerance to the presence of retrograde flow, then retrograde flow is established only during placement of a filter in the ICA distal to the plaque P. Retrograde flow is then ceased while an interventional procedure is performed on the plaque P. Retrograde flow is then re-established while the filter is removed. In another embodiment, a filter is places in the ICA distal of the plaque P and retrograde flow is established while the filter is in place. This embodiment combines the use of a distal filter with retrograde flow.

While this specification contains many specifics, these should not be construed as limitations on the scope of an invention that is claimed or of what may be claimed, but rather as descriptions of features specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or a variation of a sub-combination. Similarly, while operations are depicted in the drawings in a particular order, this should not be understood as requiring that such operations be performed in the particular order shown or in sequential order, or that all illustrated operations be performed, to achieve desirable results.

Although embodiments of various methods and devices are described herein in detail with reference to certain versions, it should be appreciated that other versions, embodiments, methods of use, and combinations thereof are also possible. Therefore the spirit and scope of the appended claims should not be limited to the description of the embodiments contained herein.

1. A vessel closure device, comprising:
an annular body defining a plane and disposed about a central axis at the center of an opening of the body, the body being movable from a generally planar configuration lying generally in the plane towards a generally cylindrical configuration extending out of the plane, the body comprising a plurality of looped elements comprising alternating first and second curved regions, the first curved regions defining an inner periphery of the body and the second curved regions defining an outer periphery of the body in the planar configuration; and
a plurality of tissue attachment features extending from the first curved regions, the attachment features being oriented generally into the opening of the body in the planar configuration in a manner that does not interfere with insertion and removal of a procedural sheath through the opening, and generally parallel to the central axis in the transverse configuration.

2. A device as in claim 1, wherein the attachment features comprise tines.

3. A device as in claim 1, wherein each of the attachment features extends along an axis that is offset from the central axis when the device is in the planar configuration.

4. A device as in claim 1, wherein each of the attachment features extends along an axis that is angled away from central axis when the device is in the planar configuration.

5. A device as in claim 1, wherein the annular body has a spring force that closes the annular body from the cylindrical configuration to the planar configuration pursuant to a generally linear rather than radial bias.

6. A device as in claim 1, wherein at least one of the attachment features extends along an axis that intersects an axis of another attachment feature when the device is in the planar configuration, and wherein none of the axes of the attachment features intersect the central axis.

7. A vessel closure device, comprising:
an annular body defining a plane and disposed about a central axis at the center of an opening of the body, the body being movable from a generally expanded configuration towards a generally compressed configuration, wherein the body is spring biased toward the compressed configuration and wherein the body applies a generally linear force to tissue as the body moves toward the compressed configuration; and
a plurality of tissue attachment features extending from the body for attaching to tissue.

8. A device as in claim 7, wherein the attachment features are oriented generally into the opening of the body in the compressed configuration in a manner that does not interfere with insertion and removal of a procedural sheath through the opening, and generally parallel to the central axis in the expanded configuration.

9. A device as in claim 7, wherein the attachment features are tines.

10. A vessel closure device, comprising:
an annular body with a central opening;
a plurality of attachment features being oriented in a manner that does not interfere with insertion and removal of a procedural sheath through the opening; and
a self-sealing member attached to the body for sealing an opening in the vessel.

11. A device as in claim 10, wherein the attachment features comprise tines.

12. A device as in claim 10, wherein the attachment features are arranged in a helical configuration.

13. A device as in claim 10, wherein the attachment features are barbed.

14. A device as in claim 10, wherein the annular body defines a plane and is disposed about a central axis at the center of an opening of the body, the body being movable from a generally planar configuration lying generally in the plane towards a generally cylindrical configuration extending out of the plane, the body comprising a plurality of looped elements comprising alternating first and second curved regions, the first curved regions defining an inner periphery of the body and the second curved regions defining an outer periphery of the body in the planar configuration

15. A device as in claim 14, wherein the attachment features extend from the first curved regions, the attachment features being oriented generally into the opening of the body in the planar configuration in a manner that does not interfere with insertion and removal of a procedural sheath through the opening, and generally parallel to the central axis in the transverse configuration

16. A device as in claim 14, wherein the annular body causes the seal member to seal the opening as the annular body moves from the cylindrical configuration to the planar configuration.

17. A vessel closure device, comprising:
an annular body defining a plane and disposed about a central axis at the center of an opening of the body, the body being movable from a generally planar configuration lying generally in the plane towards a generally cylindrical configuration extending out of the plane, the body comprising a plurality of looped elements comprising alternating first and second curved regions, the first curved regions defining an inner periphery of the body and the second curved regions defining an outer periphery of the body in the planar configuration;
a plurality of posts extend from the annular body in a cork-screw configuration;
a seal member on the posts for sealing an opening in the vessel; and
a plurality of attachment features extending from the first curved regions, wherein the posts fold as the annular body transitions from the cylindrical configuration to the planar configuration in a manner that causes the seal member to collapse in a contractile circular manner over the opening in the tissue.

18. A device as in claim 17, wherein the seal member collapses in an iris fashion.

19. A vessel closure device, comprising:
at least one clip with at least one attachment feature that attaches to tissue;
at least one closing suture pre-attached to the clip, wherein the closing suture can be tightened to cause the clip to collapse and thereby close the an opening in the tissue to which the clip is attached.

20. A device as in claim 19, wherein the suture is threaded through at least one eyelet in the clip.

21. A device as in claim 19, wherein the device includes at least two clips and wherein the closing suture is attached to at least two of the clips.

22. A vessel closure device, comprising:
an annular body with a central opening;
a plurality of attachment features being oriented in a manner that does not interfere with insertion and removal of a procedural sheath through the opening; and
a seal member attached to the body for sealing an opening in the tissue, the seal member being movable from a first position that does not interfere with the opening in the annular body and a second position that extends over the opening and seals an opening in the vessel.

23. A device as in claim 22, wherein the seal member is integral with the annular body.

24. A device as in claim 22, wherein the annular body defines a plane and is disposed about a central axis at the center of an opening of the body, the body being movable from a generally planar configuration lying generally in the plane towards a generally cylindrical configuration extending out of the plane, the body comprising a plurality of looped elements comprising alternating first and second curved regions, the first curved regions defining an inner periphery of the body and the second curved regions defining an outer periphery of the body in the planar configuration, and wherein the attachment features extend from the first curved regions, the attachment features being oriented generally into the opening of the body in the planar configuration in a manner that does not interfere with insertion and removal of a procedural sheath through the opening, and generally parallel to the central axis in the transverse configuration.

25. A device as in claim 22, further comprising a retainer removably attached to the annular body for retaining the seal member in the first position.

26. A device as in claim 25, further comprising a tether attached to the retainer for removing the retainer from the annular body so that the seal member can move to the second position.

27. A vessel closure device, comprising:
an annular body with at least one attachment feature that attaches to tissue;
a seal member fastened to the annular body for sealing an opening in the tissue;
a fastener element integral to the annular body that fastens the seal to the tissue.

28. A device as in claim 27, wherein the annular body is adapted to provide a closing force to an opening in the tissue.

29. A device as in claim 27, wherein the fastener element may be in an open state during procedural sheath insertion and removal and a closed state during fastening of the seal.

30. A device as in claim 29, further comprising a retainer for holding the fastener elements in the open state.

31. A device as in claim 30, further comprising a tether attached to the retainer for removing the retainer from the fastener elements.

32. A device as in claim 27, wherein the fastener element is at least one prong extending from the body wherein the prong fastens the seal to the tissue.

33. A device as in claim 27, wherein the annular body defines a plane and is disposed about a central axis at the center of an opening of the body, the body being movable from a generally planar configuration lying generally in the plane towards a generally cylindrical configuration extending out of the plane, the body comprising a plurality of looped elements comprising alternating first and second curved regions, the first curved regions defining an inner periphery of the body and the second curved regions defining an outer periphery of the body in the planar configuration.

34. A device as in claim 33, wherein the fastener element is a second annular body disposed above the first annular body, the second body being movable from a generally planar configuration towards a generally cylindrical configuration and wherein the fastener element fastens the seal to the tissue when in the planar configuration.

35. A device as in claim 30, further comprising an elongate tube that attaches to the retainer for removing the retainer from the fastener elements.

36. A device as in claim 35, wherein the tube is premounted on a procedural sheath.

37. A device as in claim 35, wherein the tube forms a passageway where the seal can be delivered to the clip.

38. A vessel closure device delivery system, comprising:
a delivery device that couples to a vessel closure clip for delivering the clip onto a blood vessel;
a suction element coupled to the delivery device, the suction element adapted to apply suction to a wall of the blood vessel when the delivery device is delivering the clip; and
a vessel closure clip.

39. A system as in claim 38, wherein the delivery device comprises a clip carrier assembly, the carrier assembly comprising an elongated member retaining the vessel closure clip in a delivereable configuration, a pusher member adapted to deploy the vessel closure clip, and an actuation element to actuate the pusher member with respect to the elongated member to deploy the clip.

40. A system as in claim 39, wherein the clip carrier assembly further comprises a cover member for retaining vessel closure clip on the elongated member during delivery, the cover member coupled to actuation means to release clip during deployment.

41. A system as in claim 38, wherein the suction element is attached to a syringe, a suction cartridge, or a suction pump.

42. A system as in claim 38, wherein the suction element secures the delivery system to the outer surface of the vessel wall by exerting a suction force onto the vessel wall.

43. A system as in claim 38, wherein the suction element gathers a region of tissue into a distal region of the delivery system.

44. A system as in claim 38, further comprising a guidewire lumen such that the device may be delivered over a guidewire positioned in the vessel to the outer surface of the vessel.

45. A system as in claim 38, wherein the suction element comprises a sheath.

46. A vessel closure device delivery system, comprising:
a delivery device that couples to a vessel closure clip for delivering the clip onto a blood vessel;
a retractable vessel locator removeably attached to the delivery device, the distal end of the vessel locator adapted to transition from a collapsed state suitable for insertion into a vessel and an expanded state that lodges against a wall of the vessel from inside the vessel; and
a vessel closure clip.

47. A system as in claim 46, wherein the delivery device comprises a clip carrier assembly, the clip carrier assembly comprising an elongated member retaining vessel closure clip in a delivereable configuration, a pusher member adapted to deploy the vessel closure clip, and an actuator to actuate the pusher member with respect to the elongated member to deploy the clip.

48. A system as in claim 47, wherein the carrier assembly further comprises a cover member for retaining vessel closure clip on the elongated member during delivery, the cover member coupled to an actuator to release the clip during deployment

49. A system as in claim 46, wherein the vessel locator is part of a locating device sized and constructed to function as a guidewire in the collapsed state, and wherein the locating device may be used to guide the delivery device to the vessel wall.

50. A system as in claim 49, wherein the delivery device may be removed from the locating device so that the locating device can be used to delivery the procedural sheath.

51. A vessel closure device delivery system, comprising:
a delivery device that couples to a vessel closure clip for delivering the clip onto a blood vessel;
a procedural sheath that couples onto the delivery device such that the procedural sheath can be advanced over or through the delivery device; and
a vessel closure clip.

52. A system as in claim 51, wherein the delivery device comprises a clip carrier assembly, the clip carrier assembly comprising an elongated member retaining vessel closure clip in a delivereable configuration, a pusher member adapted to deploy the vessel closure clip, and a means to actuate the pusher member with respect to the elongated member to deploy the clip.

53. A system as in claim 52, wherein the clip carrier assembly further comprises a cover member for retaining vessel closure clip on the elongated member during delivery, the cover member coupled to actuation means to release clip during deployment.

54. A system as in claim 52, wherein the delivery device further comprises a vessel locating element having a distal end adapted to transition from a collapsed state suitable for insertion into a vessel and an expanded state that lodges against a wall of the vessel from inside the vessel.

55. A system as in claim 51 wherein the vessel closure clip is premounted on the procedural sheath.

56. A system as in claim 51, wherein the procedural sheath is premounted onto the delivery device.

57. A system as in claim 51, wherein the procedural sheath includes a sheath retention element.

58. A system as in claim 51, wherein the procedural sheath includes an expandable vessel occlusion element.

59. A system as in claim 58, wherein the expandable vessel occlusion element is an inflatable balloon.

60. A system as in claim 51, wherein the sheath includes a Y-arm connection to a flow line having a lumen, the Y-arm and flow line lumens connected to the sheath so that blood flowing into the distal end of the sheath can flow through the Y-arm and into the lumen of the flow line.

61. A system as in claim 60, wherein the sheath includes a proximal extension tube having a distal end, a proximal end, and a lumen therebetween, wherein the distal end of the proximal extension is connected to the proximal end of the sheath at a junction so that the lumens of each are contiguous.

62. A system as in claim 61, wherein the proximal extension is removably connected to the proximal end of the sheath, and further comprising a hemostasis valve on the distal sheath, at a connection site of the proximal extension tube to the sheath.

63. A system as in claim 51, further comprising a guidewire lumen such that the device may be delivered over a guidewire positioned in the vessel to the outer surface of the vessel.

64. A vessel closure device delivery system, comprising:
a delivery device that couples to a vessel closure clip for delivering the clip onto a blood vessel;
a counter traction element that prevents the clip from being detached from the blood vessel during removal of the delivery device; and
a vessel closure clip.

65. A system of devices for treating carotid or cerebral artery disease or the brain, comprising:
a vessel closure clip;
a delivery device that couples to the vessel closure clip for delivering the clip onto a blood vessel; and
an arterial access sheath adapted to be introduced into a common carotid, internal carotid, or vertebral artery through a penetration in the artery and receive blood from the artery, wherein the arterial access sheath couples onto the delivery device such that the arterial access sheath can be advanced over or through the delivery device.

66. A system of devices as in claim 65, further comprising:
a treatment device adapted to be introduced into the artery through the arterial access sheath and configured to treat the carotid or cerebral artery or brain.

67. A system of devices as in claim 66, wherein the treatment device comprises an embolic system which delivers an embolic coil, material or fluid composition.

68. A system of devices as in claim 66, wherein the treatment device comprises a stent delivery catheter.

69. A system of devices as in claim 66, wherein the treatment device comprises a balloon dilatation catheter.

70. A system of devices as in claim 66, wherein the treatment device comprises a balloon occlusion catheter.

71. A system of devices as in claim 66, wherein the treatment device comprises a microcatheter that delivers a therapeutic agent.

72. A system of devices as in claim 66, wherein the treatment device comprises a thrombus disruption or removal system.

73. A system of devices as in claim 66, wherein the treatment device comprises a diagnostic angiography catheter.

74. A system of devices as in claim 66, wherein the treatment device comprises a brain tumor treatment device.

75. A system of devices as in claim 66, further comprising a shunt fluidly connected to the arterial access sheath, wherein the shunt provides a pathway for blood to flow from the arterial access sheath to a return site, and a treatment device adapted to be introduced into the artery through the arterial access sheath and configured to treat the carotid or cerebral artery or brain.

76. A system of devices as in claim 75, further comprising a flow control assembly coupled to the shunt and adapted to regulate blood flow through the shunt.

77. A system of devices as in claim 75, wherein the treatment device comprises an embolic system which delivers an embolic coil, material or fluid composition.

78. A system of devices as in claim 75, wherein the treatment device comprises a stent delivery catheter.

79. A system of devices as in claim 75, wherein the treatment device comprises a balloon dilatation catheter.

80. A system of devices as in claim 75, wherein the treatment device comprises a balloon occlusion catheter.

81. A system of devices as in claim 76, wherein the treatment device comprises a microcatheter that delivers a therapeutic agent.

82. A system of devices as in claim 75, wherein the treatment device comprises a thrombus removal or disruption system.

83. A system of devices as in claim 75, wherein the treatment device comprises a diagnostic angiography catheter.

84. A system of devices as in claim 75, wherein the treatment device comprises a brain tumor treatment device.

85. A method for closing an opening in a wall of a body lumen, comprising:
placing a clip on the wall of the body lumen;
advancing a procedural sheath through the clip into the body lumen; and
inserting a procedural device through the procedural sheath into the body lumen.

86. A method as in claim 85, further comprising:
performing a procedure using the procedural device; and
removing the procedural sheath from the clip and the body lumen.

87. A method as in claim 85, wherein the clip substantially closes the opening in the wall of the body lumen, and wherein the clip translates into a substantially planar configuration from a cylindrical configuration.

88. A method as in claim 85, wherein the procedural sheath is advanced transcervically through the clip into the body lumen.

89. A method as in claim 85, wherein the body lumen is the carotid artery.

90. A method as in claim 86, wherein the procedure comprises a carotid artery stenting procedure, an acute stroke treatment procedure, or an intracerebral procedure.

91. A method as in claim 86, wherein the procedural sheath includes an expandable vessel occlusion element, and further comprising the step of expanding the vessel occlusion element to occlude the artery.

92. A method as in claim 91, wherein the expandable vessel occlusion element is an inflatable balloon, and the step of expanding the vessel occlusion element comprising inflating the balloon.

93. A method as in claim 85, wherein the procedural sheath includes a Y-arm connection to a flow line, and further comprising the step of connecting the sheath to a reverse flow shunt.

94. A method as in claim 85, further comprising locating the wall of the body lumen using a suction element.

95. A method as in claim 85, further comprising locating the wall of the body lumen using a vessel locating device.

96. A method as in claim 95, further comprising using the vessel locating device to insert the procedural sheath.

97. A method as in claim 86, wherein the clip is attached to a pre-attached suture and further comprising tightening or tying off the suture to close the opening in the wall of the body lumen after removing the procedural sheath.

98. A method as in claim 85, wherein a self-sealing element is attached to the clip and wherein the procedural sheath is advanced through the self-sealing element

99. A method as in claim 86, wherein the clip is spring-loaded to apply a closure force to wall of the body lumen and includes a retaining element that maintains the clip in an open state and further comprising removing retaining feature after removing the sheath to permit the closure force to close the opening in the wall of the body lumen.

100. A method as in claim 99, wherein the retaining element is attached to a tether and removing the retaining feature comprises pulling on the tether.

101. A method as in claim 99, wherein removal of the retaining element comprises advancing an elongate tube which engages the retaining element, and then retracting the tube and retaining element.

102. A method as in claim 101, wherein the elongate tube is pre-mounted on the procedural sheath and further comprising removing the retaining feature while the procedural sheath is positioned through the opening in the wall of the body lumen.

103. A method as in claim 86, wherein the clip includes a fastening element and a sealing element, and further comprising fastening a sealing element to wall of the body lumen using the fastening element after removing the procedural sheath.

104. A method as in claim 103, wherein the fastening element is initially retained by a retaining element in an open position and further comprising releasing the retaining element after removal of the procedural sheath to permit the fastening feature to close on the sealing element.

105. A method as in claim 104, wherein releasing the retaining element comprises pulling on a tether

106. A method as in claim 104, wherein releasing the retaining element comprises advancing an elongate tube which attaches to the retaining element and then retracting the tube and retaining element and further comprising delivering the sealing element through the tube holding the sealing element in place with a pusher element while the tube and retaining element is removed, and then removing the pusher element.

107. A method as in claim 104, wherein an elongate tube is used to retain the fastening element in the open position and further comprising delivering the sealing element through the tube, holding the sealing element in place with a pusher element while the tube is removed, and then removing the pusher element.

108. A method as in claim 86, wherein the body lumen is the carotid artery and the procedure comprises a carotid artery stenting procedure, an acute stroke treatment procedure, or an intracerebral procedure.

109. A method as in claim 108, further comprising the step of occluding the artery after advancing the sheath into the body lumen.

110. A method as in claim 109, further comprising allowing retrograde blood flow from the artery into the sheath and from the sheath via a flow path to a return site.

111. A method as in claim 85, wherein the clip is premounted on the sheath such that the sheath serves as a central delivery shaft for the clip.

112. A method as in claim 111, wherein the clip is placed on the wall of the body lumen by pushing the clip over the sheath toward the body lumen and onto the body lumen such that the clip is placed on the wall of the body lumen after the sheath is advanced into the body lumen.

113. A method for closing an opening in a wall of a body lumen, comprising:
providing a procedural sheath having a vessel closure clip pre-mounted on the procedural sheath;
placing the procedural sheath through the wall of the body lumen;
inserting a procedural device through the sheath into the body lumen;
performing a procedure using the procedural device;
advancing the vessel closure clip; and
removing the procedural sheath from the clip and the body lumen.

114. A method as in claim 113, wherein the body lumen is the carotid artery and the procedure comprises a carotid artery stenting procedure, an acute stroke treatment procedure, or an intracerebral procedure.

115. A method as in claim 113, wherein the body lumen is the carotid artery and the procedure comprises a carotid artery stenting procedure, an acute stroke treatment procedure, or an intracerebral procedure.

116. A method as in claim 115, further comprising the step of occluding the artery after advancing the sheath into the body lumen.

117. A method as in claim 116, further comprising allowing retrograde blood flow from the artery into the sheath and from the sheath via a flow path to a return site.

118. A method for closing an opening in a wall of a body lumen, comprising:
providing a vessel closure clip delivery device with a pre-mounted procedural sheath;
placing a clip on the wall of the body lumen;
advancing the procedural sheath through the clip and through the wall of the body lumen;
inserting a procedural device through the sheath into the body lumen;
performing a procedure using the procedural device;
removing the procedural sheath from the clip and the body lumen.

119. A method as in claim 118, wherein the body lumen is the carotid artery and the procedure comprises a carotid artery stenting procedure, an acute stroke treatment procedure, or an intracerebral procedure.

120. A method as in claim 119, further comprising the step of occluding the artery after advancing the sheath into the body lumen.

121. A method as in claim 120, further comprising allowing retrograde blood flow from the artery into the sheath and from the sheath via a flow path to a return site.

122. A method for performing a procedure on a carotid or cerebral artery, comprising:
inserting a procedural sheath through the wall of the common carotid artery;
occluding the common carotid artery;
inserting a procedural device through the procedural sheath into the common carotid artery and performing a procedure on the carotid or cerebral artery;
removing the procedural sheath; and
placing a vessel closure clip on the wall of the artery to close the access site of the common carotid artery.

123. A method as in claim 122, wherein the procedural sheath includes an expandable vessel occlusion element, and the step of occluding the common carotid artery comprises expanding the vessel occlusion element.

124. A method as in claim 123, wherein the expandable vessel occlusion element is an inflatable balloon, and the step of expanding the vessel occlusion element comprises inflating the balloon.

125. A method as in claim 122, wherein the procedural sheath includes a Y-arm connection to a flow line, and further comprising the step of connecting the sheath to a reverse flow shunt.

126. A method as in claim 122, wherein the procedural sheath includes a sheath retention element, and further comprising the step of actuating the retention element after inserting the sheath to prevent inadvertent sheath removal.

127. A method as in claim 122, wherein the step of placing the vessel closure clip to close the access site of the common carotid artery comprises:
inserting the distal end of a vessel locator element into the access site of the common carotid artery and engaging the artery wall;
positioning a distal region of a clip carrier assembly adjacent to the wall, the distal region of carrier assembly configured to retain a vessel closure clip within the carrier assembly and the carrier assembly including an element to deploy the vessel closure clip into artery wall;
distally deploying the vessel closure clip from the carrier assembly such that the clip engages the vessel wall whereby the opening of the access site is drawn substantially closed.

128. A method for closing an opening in a wall of a body lumen, comprising:
placing a clip on a penetration that extends through the wall of the body lumen;
advancing a procedural sheath through the penetration into the body lumen; and
inserting a procedural device through the procedural sheath into the body lumen.

129. A method as in claim 128, wherein the body lumen is a common carotid artery and further comprising:
forming a penetration at the neck of a patient in order to access the body lumen;
allowing retrograde blood flow from the artery into the sheath and from the sheath via a flow path to a return site.

130. A method as in claim 129, further comprising using the procedural device to insert a stent in the carotid artery.

131. A method as in claim 128, wherein the clip is placed on the penetration after the procedural sheath is advanced through the penetration.

132. A method as in claim 131, wherein the clip is premounted on the sheath such that the sheath serves as a central delivery shaft for the clip.

133. A method as in claim 132, wherein the clip is placed on the penetration by pushing the clip over the sheath toward the body lumen and onto the penetration.

134. A method as in claim 131, wherein the body lumen is a common carotid artery and further comprising:
forming a penetration at the neck of a patient in order to access the body lumen;
allowing retrograde blood flow from the artery into the sheath and from the sheath via a flow path to a return site.

135. A method as in claim 128, wherein the clip is placed on the penetration prior to advancing a procedural sheath through the penetration.

136. A method as in claim 135, wherein the body lumen is a common carotid artery and further comprising:
forming a penetration at the neck of a patient in order to access the body lumen;
allowing retrograde blood flow from the artery into the sheath and from the sheath via a flow path to a return site.

137. A method as in claim 128, further comprising removing the sheath from the body lumen and wherein the clip is placed after the sheath is removed.

138. A method as in claim 137, wherein the body lumen is a common carotid artery and further comprising:
forming a penetration at the neck of a patient in order to access the body lumen;
allowing retrograde blood flow from the artery into the sheath and from the sheath via a flow path to a return site.
